(19) United States
(12) Patent Application Publication
(10) Pub. No.: US 2023/0203163 A1
(43) Pub. Date: Jun. 29, 2023

(54) COMPOSITIONS AND METHODS FOR CO-POTENTIATION OF CD3 TO TREAT A VIRAL INFECTION AND INCREASE THE IMMUNE RESPONSE AGAINST A VIRAL ANTIGEN

(71) Applicant: The Curators of the University of Missouri, Columbia, MO (US)

(72) Inventors: Diana Gil-Pages, Columbia, MO (US); Adam G. Schrum, Columbia, MO (US)

(21) Appl. No.: 18/050,845

(22) Filed: Oct. 28, 2022

Related U.S. Application Data
(60) Provisional application No. 63/273,562, filed on Oct. 29, 2021.

(51) Int. Cl. C07K 16/28; A61P 31/12

(52) U.S. Cl. CPC C07K 16/2809; A61P 31/12; C07K 2317/24; C07K 2317/35

(57) ABSTRACT

The present disclosure is generally directed to compositions and methods for treating viral infections. In particular, pharmaceutical compositions of the present disclosure include a monovalent anti-CD3 antibody and a viral antigen and their use as adjuvants to treat a viral infection and to increase the immune response produced against a viral antigen.

### STATEMENT AS TO FEDERALLY SPONSORED RESEARCH

This invention was made with government support under U01 CA244314, R01 AI097187, and R01 GM103841 awarded by National Institutes of Health. The government has certain rights in the invention.

### CROSS-REFERENCE TO RELATED APPLICATIONS

This application claims priority to U.S. Provisional Pat. Application No. 63/273,562, filed on Oct. 29, 2021, which is hereby incorporated by reference in its entirety.

### INCORPORATION OF SEQUENCE LISTING

A paper copy of the Sequence Listing and a computer readable form of the Sequence Listing containing the file named “22UMC021_Sequence_Listing_ST25”, which is 10,545 bytes in size (as measured in MICROSOFT WINDOWS® EXPLORER), are provided herein and are herein incorporated by reference. This Sequence Listing consists of SEQ ID NOs: 1-53.

### BACKGROUND

The present disclosure is directed to compositions and methods for use in viral infections in a subject in need thereof. In particular, the present disclosure is directed to pharmaceutical compositions comprising a monovalent anti-CD3 antibody and one of a viral antigen and a nucleic acid that expresses the viral antigen. The present disclosure is further directed to methods using these pharmaceutical compositions to treat a viral infection in a subject in need thereof and to increase an immune response produced against a viral antigen in a subject in need thereof.

Herpes human cytomegalovirus (HCMV) infects the population at high incidence. Infection is often asymptomatic and controlled by long-lasting T-cell responses driving the virus to latency, although sterilizing immunity is not induced. Chronic inflammation or immune compromise can allow HCMV reactivation and lifethreatening disease. Thus, there is great interest in developing new immune-boosting therapies to treat/prevent HCMV recurrence.

The inventors previously developed an immunostimulatory concept, CD3 copotentiation. It was demonstrated that an anti-mouse-CD3 mono-Fab fragment whose binding was functionally inert if T cells encountered no antigen and did not inhibit T cells stimulated by strong antigens, but if T cells were stimulated by weak antigens, then coincident Fab-CD3 engagement improved various responses elicited from naive CD8 T cells (Hoffmann et al., 2015, Sci. Adv. 1(9):e1500415). In vivo, the Fab reduced tumor burden of B16-F10 melanoma by a mechanism dependent on CD4 and CD8 T cells and T-cell antigen receptor (TCR) antigen specificity, and the anti-CD3 mono-Fab induced a stimulation-poised CD3 conformation thought to amplify signaling upon weak antigen engagement by TCR (Gil, et al., 2005, J Exp Med. 201(4): 517-522; de la Cruz, et al., 2011, J Immunol 186(4):2282-2290).

As provided in the present disclosure, human T-cell copotentiation can increase the expansion of different classes of T-cell clones responding to recall antigens of different strengths. Thus, CD3 copotentiation can provide therapeutic treatment for chronic, persistent viral infection.

### SUMMARY

The present disclosure provides compositions and methods directed to using monovalent anti-CD3 antibodies as adjuvants to increase the immune response produced against a viral antigen. In particular, the present disclosure provides pharmaceutical compositions including a monovalent anti-CD3 antibody and either a viral antigen or a nucleic acid that expresses the viral antigen. The present disclosure is further directed to methods of treating a viral infection using the pharmaceutical compositions and methods of increasing a subject’s immune response against a viral antigen using the pharmaceutical compositions.

One aspect of the present disclosure provides pharmaceutical compositions comprising a monovalent anti-CD3 antibody, wherein the monovalent anti-CD3 antibody specifically binds to CD3, induces a conformational change in a CD3 complex (CD3Δc), does not initiate CD3 signaling, does not block interaction of a T cell receptor with a viral antigen, and does not block a T cell’s signaling response to the viral antigen; and at least one of the viral antigen and a nucleic acid that encodes the viral antigen.

A further aspect of the present disclosure is a method of treating a viral infection in a subject having or suspected of having a viral infection. The method includes administering to the subject a pharmaceutical composition, the pharmaceutical composition including a monovalent anti-CD3 antibody, wherein the monovalent anti-CD3 antibody specifically binds to CD3, induces a conformational change in a CD3 complex (CD3Δc), does not initiate CD3 signaling, does not block interaction of a T cell receptor with a viral antigen, and does not block a T cell’s signaling response to the viral antigen; and at least one of the viral antigen and a nucleic acid that encodes the viral antigen.

Another aspect of the present disclosure is a method for increasing an immune response to a viral antigen in a subject in need thereof. The method includes administering a pharmaceutical composition including a monovalent anti-CD3 antibody, wherein the monovalent anti-CD3 antibody specifically binds to CD3, induces a conformational change in a CD3 complex (CD3Δc), does not initiate CD3 signaling, does not block interaction of a T cell receptor with the viral antigen, and does not block a T cell’s signaling response to the viral antigen; and at least one of the viral antigen and a nucleic acid that encodes the viral antigen to the subject, wherein the subject produces an immune response against the viral antigen.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts how mono-OKT3-Fab and mono-UCHT1-Fab bind T cells, detected by positive secondary anti-Ms-IgG staining by flow cytometry of OT-I.JRT3 cells and primary human CD4 and CD8 T cells from PBMCs.

FIG. 1B depicts how mono-OKT3-Fab does not block TCR-antigen binding, in contrast to mono-UCHT1-Fab. OT-I.JRT3 or CD8 T cells isolated from PBMCs that were previously expanded with NLV peptide were preincubated with indicated immunoglobulins and stained for binding of Kb/OVA-tetramer (left) or A2/NLV-tetramer (right), respectively.

FIG. 1C depicts how mono-OKT3-Fab does not impair the T-cell response to cognate antigen, unlike mono-UCHT1-Fab. OT-I.JRT3 cells were cultured with null peptide (pFARL) or antigenic peptide (pOVA) presented on T2-Kb APCs in the presence of indicated immunoglobulins and analyzed for CD69 upregulation and TCR downregulation. Frequencies of CD69(+) and V85(+) cells are shown (mean ± SD from triplicate samples, 2-tailed unpaired Student t test).

FIG. 1D depicts how binding of mono-Fabs does not stimulate T cells in the absence of antigen. PBMCs were incubated with indicated immunoglobulins, after which CD4 and CD8 T cells were analyzed for the induction of surface CD69 and intracellular Nur77 by flow cytometry. Frequencies of CD69(+) and Nur77(+) T cells are shown (mean ± SD from triplicate samples, 2-tailed unpaired Student t test).

FIG. 1E depicts how binding of mono-Fabs does not stimulate T cells in the absence of antigen. PBMCs were incubated with indicated immunoglobulins in the presence or absence of pervanadate (PV). Phosphotyrosine was detected by western blot (WB) of equivalent cell lysates.

FIG. 1F depicts how mono-Fabs induce CD3Ac. PBMC lysates were incubated with APA1/1 (to block CD3 pull-down), Ms-IgG-Fab (to reveal basal level of CD3Ac), or mono-Fabs (test conditions) and assessed for induction of CD3Ac by the CD3 pull-down assay. Post-CD3Ac open conformation was detected with anti-CD3C by western blot. Inducible CD3Ac is measured by fold-increase over basal level. TL, total lysate before pull-down. Data are representative of ≥3 independent experiments. Ms-IgG-Fab, negative control.

FIG. 2A depicts how PBMCs were cultured with or without exogenous NLV peptide in the presence of mono-OKT3-Fab or control Ms-IgG-Fab. On day 9, cells were analyzed by flow cytometry for the number of A2/NLV-tetramer(+) CD8 T cells from exog-NLV-bulk-responsive donors (mean ± SD, 2-tailed paired Student t test). Each symbol represents the average of ≥3 independent experiments per donor.

FIG. 2B depicts how PBMCs were cultured with or without exogenous NLV peptide in the presence of mono-OKT3-Fab or control Ms-IgG-Fab. Mono-OKT3-Fab increased the production of IFN-g, as measured by ELISA of day 7 supernatants (mean ± SD, 1-tailed paired Student t test). Each symbol represents the average of ≥3 independent experiments per donor.

FIG. 2C depicts how PBMCs were cultured with or without exogenous NLV peptide in the presence of mono-OKT3-Fab or control Ms-IgG-Fab. Mono-OKT3-Fab increased the production of granzyme B, as measured by ELISA of day 7 supernatants (mean ± SD, 1-tailed paired Student t test). Each symbol represents the average of ≥3 independent experiments per donor.

FIG. 2D depicts how CD8 T-cell isolates (effectors) were cultured at the indicated effector to target ratios with NLV-loaded CD4 T cells (targets) overnight and analyzed for specific lysis of targets (mean ± SD of duplicates).

FIG. 3A depicts how PBMCs were cultured with or without exogenous NLV peptide in the presence of mono-OKT3-Fab or control Ms-IgG-Fab for 9 days as in FIG. 2A. Counts of A2/NLV-tetramer(-) CD8 T cells are shown for exog-NLV-bulk-responsive donors. Each symbol represents the average of ≥3 independent experiments per donor (mean ± SD, 2-tailed paired Student t test).

FIG. 3B depicts how (A-B) PBMCs were cultured with or without exogenous NLV peptide in the presence of mono-OKT3-Fab or control Ms-IgG-Fab for 9 days as in FIG. 2A. Counts of A2/NLV-tetramer(-) CD8 T cells are shown for exog-NLV-bulk-nonresponsive donors. Each symbol represents the average of ≥3 independent experiments per donor (mean ± SD, 2-tailed paired Student t test).

FIG. 3C depicts how PBMCs were cultured with or without exogenous NLV peptide in the presence of Ms-IgG-Fab (control), mono-OKT3-Fab, or mono-UCHT1-Fab for 9 days. Mono-UCHT1-Fab dampened the copotentiation effect as compared with mono-OKT3-Fab in A2/NLV-tetramer(+) CD8 T cells. One representative experiment of donor 47M is shown for 3 replicates (mean ± SD from triplicate samples, 2-tailed unpaired Student t test).

FIG. 3D depicts how PBMCs were cultured with or without exogenous NLV peptide in the presence of Ms-IgG-Fab (control), mono-OKT3-Fab, or mono-UCHT1-Fab for 9 days. Mono-UCHT1-Fab dampened the copotentiation effect as compared with mono-OKT3-Fab in A2/NLV-tetramer(-) CD8 T cells. One representative experiment of donor 47M is shown for 3 replicates (mean ± SD from triplicate samples, 2-tailed unpaired Student t test).

FIG. 3E depicts how PBMCs were cultured with or without exogenous NLV peptide in the presence of Ms-IgG-Fab or mono-OKT3-Fab and with or without the CD8 blocking antibody, DK25, for 7 days. Blocking CD8 reduced the copotentiation effect of mono-OKT3-Fab for A2/NLV-tetramer(+) CD8 T cells. One representative experiment of donor 47M is shown for 3 replicates (mean ± SD from triplicate samples, 2-tailed unpaired Student t test).

FIG. 3F depicts how PBMCs were cultured with or without exogenous NLV peptide in the presence of Ms-IgG-Fab or mono-OKT3-Fab and with or without the CD8 blocking antibody, DK25, for 7 days. Blocking CD8 reduced the copotentiation effect of mono-OKT3-Fab for A2/NLV-tetramer(-) CD8 T cells. One representative experiment of donor 47M is shown for 3 replicates (mean ± SD from triplicate samples, 2-tailed unpaired Student t test).

FIG. 4A depicts how TCR clones for exog-NLV-bulk-responsive donor 72F were ranked from most abundant to least abundant for each condition. Differences in rank-vs-rank performance concentrated in the top 10 clones.

FIG. 4B depicts how TCR clones for exog-NLV-bulk-responsive donor 53M were ranked from most abundant to least abundant for each condition. Differences in rank-vs-rank performance concentrated in the top 10 clones.

FIG. 4C depicts how TCR clones for exog-NLV-bulk-responsive donor 28M were ranked from most abundant to least abundant for each condition. Differences in rank-vs-rank performance concentrated in the top 10 clones.

FIG. 4D depicts how TCR clones for exog-NLV-bulk-responsive donor 47M were ranked from most abundant to least abundant for each condition. Differences in rank-vs-rank performance concentrated in the top 10 clones.

FIG. 4E depicts how among top-ranked clones, there was variability in the extent to which clones were amplified by exogenous NLV, mono-OKT3-Fab, or both in combination. The top 10 ranked clones for exog-NLV-bulk-responsive donor 72F from the NLV + mono-OKT3-Fab condition are shown with their corresponding live-cell number abundance in the other 3 conditions. Boxed amino acid sequences indicate NLV-specific clones (clones with greater abundance in NLV + Ms-IgG-Fab versus no exogenous peptide + Ms-IgG-Fab condition or, for public TCRs, observation of that pattern in ≥1 other donor or previously reported in the literature). Sequences in bold represent public TCR-bearing clones appearing in multiple donors in the present study or previously reported in the literature. Heatmaps visualize the increase in clonal cell number generated by exogenous NLV, mono-OKT3-Fab, or both in combination.

FIG. 4F depicts the same as FIG. 4E, except the top 10 ranked clones are for exog-NLV-bulk-responsive donor 53M.

FIG. 4G depicts the same as FIG. 4E, except the top 10 ranked clones are for exog-NLV-bulk-responsive donor 28M.

FIG. 4H depicts the same as FIG. 4E, except the top 10 ranked clones are for exog-NLV-bulk-responsive donor 47M.

FIG. 5A depicts how among top NLV-specific clones, those from exog-NLV-bulk-responsive donors respond more than those from exog-NLV-bulk-nonresponsive donors to exogenous NLV. NLV-specific fold increase in TCR abundance was determined for gold-response clones from exog-NLV-bulk-responsive donors versus those from nonresponsive donors. Each dot represents the fold-increase of a TRBV-CDR3-bearing clone (mean ± SEM, 1-tailed unpaired Student t test).

FIG. 5B depicts how NLV-specific fold-increase in TCR abundance was also assessed when gold-response clones from both types of donors were cultured in the presence of mono-OKT3-Fab. Each dot represents the fold-increase of a TRBV-CDR3-bearing clone (mean ± SEM, 1-tailed unpaired Student t test).

FIG. 5C depicts how exog-NLV-bulk-nonresponsive donors respond more than exog-NLV-bulk-responsive donors to CD3 copotentiation when it is driven by exogenous NLV. Mono-OKT3-Fab-specific fold increase in TCR abundance was determined for gold-response clones from exog-NLV-bulk-responsive donors versus those from nonresponsive donors. Data are included for gold-response clones in exog-NLV-bulk-responsive and nonresponsive donors. Each dot represents the fold-increase of a TRBV-CDR3-bearing clone (mean ± SEM, 1-tailed unpaired Student t test).

FIG. 6 depicts different T-cell clonal signatures of maximal recall response to NLV when providing copotentiation with anti-CD3 mono-Fab. As shown by private TCR clone NLV:HLA-A2 immunodominant on the left, maximum recall response of private immunodominant TCR clones to exogenous NLV is mainly caused by the peptide (arrow, bottom segment), with a smaller contribution coming from copotentiation delivered by anti-CD3 mono-Fab (arrow, top segment). As shown by public TCR clone NLV:HLA-A2 immunodominant in the middle, maximum recall immunodominant response of public TCR clones to NLV is driven by either (1) copotentiation (left arrow, top segment), with the smallest contribution from natural amounts of NLV presented in HCMV(1) APCs; or (2) exogenous NLV alone (right arrow). As shown by weak TCR clone NLV:H LA-other not immunodominant on the right, NLV weak TCR clones reach their maximum recall response to exogenous NLV mainly by copotentiation (arrow, top segment), with a smaller contribution coming from exogenous NLV peptide (arrow, bottom segment). Created with BioRender.

FIG. 7A depicts preparation of purified monovalent Fabs via papain digestion.

FIG. 7B depicts binding of purified monovalent Fabs to CDR complex.

### DETAILED DESCRIPTION

The present disclosure is directed to compositions and methods using monovalent anti-CD3 antibodies and viral antigens for treating viral infections and increasing immune response to viral infection. In particular, the present disclosure provides pharmaceutical compositions comprising a monovalent anti-CD3 antibody and at least one of the viral antigen or a nucleic acid that encodes the viral antigen. The present disclosure further provides methods for treating a viral infection in a subject in need thereof and for increasing immune response produced against a viral antigen in a subject in need thereof.

In one aspect, the present disclosure is directed to a pharmaceutical composition including a monovalent anti-CD3 antibody, wherein the monovalent anti-CD3 antibody induces a conformational change in a CD3 complex (CD3Δc) and does not initiate CD3 signaling, does not block interaction of a T cell receptor with a viral antigen, and does not block a T cell’s signaling response to the viral antigen; and at least one of the viral antigen or a nucleic acid that encodes the viral antigen. The monovalent anti-CD3 antibodies provided herein can bind to a CD3 dimer with little or no detectable binding to a CD3 polypeptide not in the form of a CD3 dimer. In some cases, the monovalent anti-CD3 antibodies provided herein can bind to a CD3γε dimer with little or no detectable binding to a CD3ε polypeptide not in the form of a CD3γε dimer and with little or no detectable binding to a CD3γ polypeptide not in the form of a CD3γε dimer. For example, monovalent anti-CD3 antibodies can bind to a human CD3γε dimer with little or no detectable binding to a human CD3ε polypeptide not in the form of a CD3γε dimer and with little or no detectable binding to a human CD3γ polypeptide not in the form of a CD3γε dimer. In some cases, the monovalent anti-CD3 antibodies provided herein can bind to a human CD3δε dimer or a chimeric mouse/human CD3δε dimer with little or no detectable binding to a CD3δ polypeptide not in the form of a CD3δε dimer and with little or no detectable binding to a CD3ε polypeptide not in the form of a CD3δε dimer. It should be understood that the viral antigen encoded by the nucleic acid is expressed. In such case, the nucleic acid can further include other elements such as promoters and expression control elements such that the viral antigen is produced.

The term “epitope” refers to an antigenic determinant on an antigen to which the paratope of an antibody binds. Epitopic determinants usually consist of chemically active surface groupings of molecules (e.g., amino acid or sugar residues) and usually have specific three dimensional structural characteristics as well as specific charge characteristics.

The term “antibody” as used herein refers to intact antibodies, digestion fragments, specified portions and variants thereof, including antibody mimetics or comprising portions of antibodies that mimic the structure and/or function of an antibody or specified fragment or portion thereof, including single chain antibodies and fragments thereof.

The term “monovalent antibody” as used herein refers to an antibody with affinity for one epitope or antigen. It has exactly one antigen-binding region comprising a heavy chain variable domain (VH) and optionally a light chain variable domain (VL). For example, Fab, Fab′, scFv, Fv, and Fd fragments and nanobodies are all monovalent antibodies, but an intact antibody and a F(ab′)2 fragment (which both contain two antigen-binding regions) are not monovalent antibodies.

As used herein “CD3 complex” means any CD3 complex of a CD3εγ heterodimer, a CD3εδ heterodimer and a ζζ homodimer. These three dimers are the CD3 complex and they associate all together with the TCRαβ heterodimer or TCR to form the TCR/CD3 complex made of CD3 subunits including CD3ε, CD3γ, CD3δ, and ζ. A monovalent anti CD3 antibody then binds to the CD3 complex. The precise binding epitope of an anti-CD3 antibody may be in any of the components of the CD3 complex. In the case of the OKT3 antibody, the binding epitope is in CD3ε when dimerized with either CD3γ or CD3δ. As a result, OKT3 binds to the two CD3 dimers, CD3εγ and CD3εδ via the epitope in the CD3ε of the CD3εγ and the CD3εδ complexes.

Suitable antibodies having the ability to bind CD3 (CD3εγ and the CD3εδ) include the OKT3 antibody, the SP34-2 antibody, the Hit3a antibody, the UCHT1 antibody, the SK7 antibody, the MEM-57 antibody, the Forlumab/28F11-AE/NI-0401 antibody, the Teplizumab/PRV-031/MGA031 antibody, the Visilizumab/HuM291 antibody, and the Otelixizumab/ChAglyCD3/TRX4 antibody. The anti-CD3 antibodies are processed as described herein to obtain the monovalent antibody (e.g., Fab fragments, F(ab′)2 fragments, Fab′ fragments, scFv fragments, Fv fragments, Fd fragments and nanobodies). Additionally, or alternatively, monovalent anti-CD3 antibodies can be produced using recombinant methods as described herein. Particularly suitable monovalent anti-CD3 antibody include a monovalent OKT3 antibody, a monovalent SP34-2 antibody, a monovalent Hit3a antibody, and a monovalent SK7 antibody. The 7D6 antibody and the 17A2 antibody specifically bind mouse CD3γε dimer, for example, thus, a monovalent 7D6 antibody and a monovalent 17A2 antibody are particularly suitable for detecting chimeric mouse/human CD3 complexes. Fab fragments, F(ab′)2 fragments, Fab′ fragments, scFv fragments, Fv fragments, Fd fragments and nanobodies from such an antibody are examples of monovalent anti-CD3 antibodies. Suitable antibodies can also include derivatives of anti-CD3 antibodies. Additional anti-CD3 antibodies useful in the present disclosure include commercially available anti-CD3 antibodies (Abcam, Cambridge, UK).

The monovalent anti-CD3 antibody can be from an anti-human CD3 antibody including from a humanized anti-human CD3 antibody and from a fully human anti-human CD3 antibody. The monovalent anti-CD3 antibody can be a monovalent anti-CD3γε antibody. The monovalent anti-CD3 antibody can comprise papain digested anti-CD3 antibody to produce Fab fragments of the anti-CD3 antibody. A particularly suitable monovalent anti-CD3 antibody is Fab fragments of monoclonal anti-CD3 antibody OKT3.

The viral antigen can be from any virus. Particularly suitable viral antigens can be from a virus causing a chronic infection. Examples include Human cytomegalovirus (HCMV), influenza, coronaviruses, rhinoviruses, HIV, hepatitis (A, B, C, D, E, G) viruses, polio virus, rabis virus, rubeola virus, variolla virus, mumps virus, papilloma virus, and herpes zoster virus.

The viral antigen can be a peptide of the viral antigen, a polypeptide of the viral antigen, and/or a nucleic acid that encodes the viral antigen. The viral antigen can comprise a polypeptide including NLVPMVATV (SEQ ID NO: 1) polypeptide. In some embodiments, the pharmaceutical composition comprises Fab fragments of a monoclonal anti-CD3 antibody and a nucleic acid that encodes NLVPMVATV (SEQ ID NO: 1) peptide. In some embodiments, the pharmaceutical composition comprises Fab fragments of a monoclonal anti-CD3 antibody and a peptide. Suitable peptides include, for example, NLVPMVATV (SEQ ID NO: 1), CASSKVTGTGNYGYTF (SEQ ID NO: 2), CASSLALNTEAFF (SEQ ID NO: 3), CASSPSTGTIYGYTF (SEQ ID NO: 4), CASSPITGQGAYGYTF (SEQ ID NO: 5), CATFEEPNEKLFF (SEQ ID NO: 6), CASSPWAYATDTQYF (SEQ ID NO: 7), CASSYADRGAGELFF (SEQ ID NO: 8), CASRDRENTEAFF (SEQ ID NO: 9), CASSIDSPNTEAFF (SEQ ID NO: 10), CARTGYEDTEAFF (SEQ ID NO: 11), CASSRTSINEQFF (SEQ ID NO: 12), CASSPEGGGGAFF (SEQ ID NO: 13), CASSPTTGTGTYGYTF (SEQ ID NO: 14), CASSLEGYTEAFF (SEQ ID NO: 15), CASSPIAGYPHEQYF (SEQ ID NO: 16), CASSPGTYGYTF (SEQ ID NO: 17), CASSIMNEQFF (SEQ ID NO: 18), CASSLAPPYEQYF (SEQ ID NO: 19), CASSQGVGLGEKLFF (SEQ ID NO: 20), CASSPRDNPNYGYTF (SEQ ID NO: 21), CASSSVNEQFF (SEQ ID NO: 22), CASSPKTGATYGYTF (SEQ ID NO: 23), CASTPQTGTGYYGYTF (SEQ ID NO: 24), CASGLGVNTEAFF (SEQ ID NO: 25), CASRDGGYEQYF (SEQ ID NO: 26), CASSSRTSGRFYEQYF (SEQ ID NO: 27), CASSLDPSGRLGDEQYF (SEQ ID NO: 28), CASSINYSNQPQHF (SEQ ID NO: 29), CASSPKTGTGTYGYTF (SEQ ID NO: 30), CSANQGGGNTEAFF (SEQ ID NO: 31), CSVTLPQADGRYGYTF (SEQ ID NO: 32), CASSSAYYGYTF (SEQ ID NO: 33), CASSNPGGSSYYEQYF (SEQ ID NO: 34), CASSQEPGNYGYTF (SEQ ID NO: 35), CASSQTRGAGNTIYF (SEQ ID NO: 36), CASSLGQAYEQYF (SEQ ID NO: 37), CASRQGFPGNEQFF (SEQ ID NO: 38), CASRSLRDLNTEAFF (SEQ ID NO: 39), CASSQVPDSDCNQPQHF (SEQ ID NO: 40), CASSEEWGTSGGANEQFF (SEQ ID NO: 41), CASCSTTGYETQYF (SEQ ID NO: 42), CASSLAETENTEAFF (SEQ ID NO: 43), CASSSRFGTGTHEQYF (SEQ ID NO: 44), CASSQDYPPAGGTNNEQFF (SEQ ID NO: 45), CSVEDEDSRTDTQYF (SEQ ID NO: 46), CSAGRGIKTGRSETQYF (SEQ ID NO: 47), CASSRQRTYTGELFF (SEQ ID NO: 48), CASSVAGGLQETQYF (SEQ ID NO: 49), CASSLVGVEAFF (SEQ ID NO: 50), CASSLQTGVAFF (SEQ ID NO: 51), and combinations thereof. A particularly suitable pharmaceutical composition is a pharmaceutical composition including Fab fragments of monoclonal antibody OKT3 and NLVPMVATV (SEQ ID NO: 1) peptide.

The pharmaceutical compositions can also further comprise a pharmaceutical excipient or adjuvant.

An intact antibody comprises two light chains and two heavy chains. Each light chain is made of two protein domains: one variable domain (VL) that includes the antigen binding site of the light chain and one constant domain (CL). Each heavy chain is made of four or five protein domains: one variable domain (VH) that includes the antigen binding site of the heavy chain and three or four constant regions (CH1, CH2, and CH3 or CH1, CH2, CH3, and CH4). These protein domains of an antibody can be divided into three fragments: two Fabs (antigen-binding fragments) and one Fc (crystallizable fragment). Each Fab comprises a light chain (VL and CL domains) and the VH and CH1 domains of a heavy chain. The Fc domain comprises the remaining constant domains (CH2, CH3, and CH4 if present) of the two heavy chains. Fab fragments can be generated via papain digestion of an intact antibody and standard recombinant molecular biology techniques.

Such monovalent antibodies as described above can be produced by enzymatic cleavage as well as synthetic or recombinant techniques, as known in the art and/or as described herein. Antibodies can also be produced in a variety of truncated forms using antibody genes in which one or more stop codons have been introduced upstream of the natural stop site. The various domains and/or fragments of antibodies can also be joined together chemically by conventional techniques, or can be prepared as a contiguous protein using genetic engineering techniques with an optional linker between domains and/or fragments.

F(ab′)2 fragments can be generated via pepsin digestion of an intact antibody and standard recombinant molecular biology techniques. F(ab′)2 fragments comprise two Fab fragments joined via disulfide bonds and a small portion of the Fc region. Partial reduction disrupts the disulfide bonds and produces two Fab′ fragments. Each Fab′ fragment, like a Fab fragment, comprises a light chain (VL and CL domains) and the VH and CH1 domains of a heavy chain, but like a F(ab′)2 fragment, it also comprises a small portion of the Fc region.

An scFv (single chain Fv) fragment comprises a fusion of a VL and a VH domain of an antibody and can be generated via standard recombinant molecular biology techniques. An Fv (variable fragment) comprises a VL and a VH domain of an antibody but can be kept intact via non-covalent interactions between the two domains rather than a fusion. Fv fragments can be generated via enzymatic digestion and standard recombinant molecular biology techniques.

An Fd fragment comprises the VH and CH1 domains of a heavy chain. An Fd fragment can be generated from an intact antibody via pepsin digestion, partial reduction, and reaggregation, and standard recombinant molecular biology techniques.

The identity and methods of generation of the above fragments (i.e. Fab, F(ab′)2, Fab′, scFv, Fv, and Fd fragments) are well known in the art (see, e.g. Colligan et al., Current Protocols in Immunology, John Wiley & Sons, NY, NY, (1994-2001)).

A nanobody (also called a called single-domain antibody (sdAb) or variable domain of the heavy chain of HCAb (VHH)) comprises one heavy chain variable domain (VH). Nanobodies (sdAbs/VHHs) can be generated via immunization of camelids and subsequent nanobody isolation and purification as well as via standard molecular biology techniques such as screening of synthetic libraries and genetic engineering techniques (see, e.g. Muyldermans, S. (2021) “A guide to: generation and design of nanobodies.” FEBS. 288(7):2084-2102).

Any appropriate method can be used to produce monovalent antibodies from intact antibodies. Antibody fragments can be prepared by proteolytic hydrolysis of an intact antibody or by the expression of a nucleic acid encoding the fragment. Antibody fragments can be obtained by pepsin or papain digestion of intact antibodies by conventional methods. For example, Fab fragments can be produced by enzymatic cleavage of antibodies with papain. In some cases, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. In some cases, an enzymatic cleavage using pepsin can be used to produce two monovalent Fab′ fragments and an Fc fragment directly. These methods are described, for example, by Goldenberg (U.S. Pat. Nos. 4,036,945 and 4,331,647). See also Nisonhoff et al., Arch. Biochem. Biophys. 89:230 (1960); Porter, Biochem. J. 73:119 (1959); Edelman et al., METHODS IN ENZYMOLOGY, VOL. 1, page 422 (Academic Press 1967); and Coligan et al. at sections 2.8.1 2.8.10 and 2.10.1 2.10.4.

Other methods of cleaving antibodies, such as separation of heavy chains to form monovalent light heavy chain fragments, further cleavage of fragments, or other enzymatic, chemical, or genetic techniques may also be used provided the fragments retain some ability to bind (e.g., selectively bind) its epitope.

The antibodies provided herein that can be used to make monovalent anti-CD3 antibodies provided herein can be any antibody (e.g., a monoclonal antibody) having binding affinity (e.g., specific binding affinity) for CD3 complex. In some cases, the antibody fragments can be made from anti-human CD3 antibodies. In some cases, the antibody fragments can be made from humanized or human origin anti-human CD3 antibodies. In some cases, the antibody fragments can have the ability to increase the immune response produced against a viral antigen.

Anti-CD3 antibodies are well known in the art and are commercially available. Examples of commercially available mouse anti-human CD3 antibodies include OKT3, UCHT1, Hit3a, SP34-2, SK7, and MEM-57. Forlumab/28F11-AE/NI-0401 is an example of a fully human anti-CD3 mAb. These antibodies can also include derivatives of other anti-CD3 antibodies. For example, Teplizumab/PRV-031/MGA031 is an example of a humanized antibody derived from moving the CDR region of OKT3 into a human antibody backbone. Visilizumab/HuM291 and Otelixizumab/ChAglyCD3/TRX4 are other examples of humanized anti-CD3 antibodies. Additional anti-CD3 antibodies useful in the present disclosure include commercially available anti-CD3 antibodies (Abcam, Cambridge, UK). A most preferred antibody is OKT3.

Kjer-Nielsen, et al. ((2004) “Crystal structure of the human T cell receptor CD3εγ heterodimer complexed to the therapeutic mAb OKT3.” PNAS, 101(20): 7675-7680) discloses the crystal structure of human CD3εγ heterodimer and how it complexes with monoclonal antibody OKT3.

Arnett, et al. ((2004) “Crystal structure of a human CD3-ε/δ dimer in complex with a UCHT1 single-chain antibody fragment.” PNAS, 101(46): 16268-16273) discloses a crystal structure of human CD3-ε/δ dimer in complex with a UCHT1 single-chain antibody fragment.

Antibodies provided herein can be prepared using any appropriate method. For example, a sample containing CD3 complex (e.g., a human CD3γε dimer, a chimeric mouse/human CD3γε dimer, a human CD3δε dimer, and a chimeric mouse/human CD3δε dimer) can be used as an immunogen to elicit an immune response in an animal such that specific antibodies are produced. The immunogen used to immunize an animal can be chemically synthesized or derived from translated cDNA. In some cases, cells (e.g., mouse T cells) transfected to express a CD3γε dimer (e.g., a human CD3γε dimer or a chimeric mouse/human CD3γε dimer) can be used as an immunogen. In some cases, the immunogen can be conjugated to a carrier polypeptide, if desired. Commonly used carriers that are chemically coupled to an immunizing polypeptide include, without limitation, keyhole limpet hemocyanin (KLH), thyroglobulin, bovine serum albumin (BSA), and tetanus toxoid.

The preparation of polyclonal antibodies is well-known to those skilled in the art. See, e.g., Green et al., Production of Polyclonal Antisera, in IMMUNOCHEMICAL PROTOCOLS (Manson, ed.), pages 1 5 (Humana Press 1992) and Coligan et al., Production of Polyclonal Antisera in Rabbits, Rats, Mice and Hamsters, in CURRENT PROTOCOLS IN IMMUNOLOGY, section 2.4.1 (1992). In addition, those of skill in the art will know of various techniques common in the immunology arts for purification and concentration of polyclonal antibodies, as well as monoclonal antibodies (Coligan et al., Unit 9, Current Protocols in Immunology, Wiley Interscience, 1994).

The preparation of monoclonal antibodies also is well-known to those skilled in the art. See, e.g., Kohler & Milstein, Nature 256:495 (1975); Coligan et al., sections 2.5.1 2.6.7; and Harlow et al., ANTIBODIES: A LABORATORY MANUAL, page 726 (Cold Spring Harbor Pub. 1988). Briefly, monoclonal antibodies can be obtained by injecting mice with a composition comprising an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein A Sepharose, size exclusion chromatography, and ion exchange chromatography. See, e.g., Coligan et al., sections 2.7.1 2.7.12 and sections 2.9.1 X.3; Barnes et al., Purification of Immunoglobulin G (IgG), in METHODS IN MOLECULAR BIOLOGY, VOL. 10, pages 79 104 (Humana Press 1992).

In addition, methods of in vitro and in vivo multiplication of monoclonal antibodies are well known to those skilled in the art. Multiplication in vitro can be carried out in suitable culture media such as Dulbecco’s Modified Eagle Medium or RPMI 1640 medium, optionally replenished by mammalian serum such as fetal calf serum, or trace elements and growth sustaining supplements such as normal mouse peritoneal exudate cells, spleen cells, and bone marrow macrophages. Production in vitro provides relatively pure antibody preparations and allows scale up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells (e.g., osyngeneic mice) to cause growth of antibody producing tumors. Optionally, the animals are primed with a hydrocarbon, especially oils such as pristane (tetramethylpentadecane) prior to injection. After one to three weeks, the desired monoclonal antibody is recovered from the body fluid of the animal.

In some cases, the antibodies provided herein can be made using non-human primates. General techniques for raising therapeutically useful antibodies in baboons can be found, for example, in Goldenberg et al., International Patent Publication WO 91/11465 (1991) and Losman et al., Int. J. Cancer, 46:310 (1990).

In some cases, the antibodies can be humanized monoclonal antibodies. Humanized monoclonal antibodies can be produced by transferring mouse complementarity determining regions (CDRs) from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions when treating humans. General techniques for cloning murine immunoglobulin variable domains are described, for example, by Orlandi et al., Proc. Nat′l. Acad. Sci. USA 86:3833 (1989). Techniques for producing humanized monoclonal antibodies are described, for example, by Jones et al., Nature 321:522 (1986); Riechmann et al., Nature 332:323 (1988); Verhoeyen et al., Science 239:1534 (1988); Carter et al., Proc. Nat′l. Acad. Sci. USA 89:4285 (1992); and Sandhu, Crit. Rev. Biotech. 12:437 (1992); Singer et al., J. Immunol. 150:2844 (1993). In some cases, humanization such as super humanization can be used as described elsewhere (Hwang et al., Methods, 36:35-42 (2005)). In some cases, SDR grafting (Kashmiri et al., Methods, 36:25-34 (2005)), human string content optimization (Lazar et al., Mol. Immunol., 44:1986-1998 (2007)), framework shuffling (Dall’Acqua et al., Methods, 36:43-60 (2005); and Damschroder et al., Mol. Immunol., 44:3049-3060 (2007)), and phage display approaches (Rosok et al., J. Biol. Chem., 271:22611-22618 (1996); Radar et al., Proc. Natl Acad. Sci. USA, 95:8910-8915 (1998); and Huse et al., Science, 246:1275-1281 (1989)) can be used to obtain anti-CD3 antibodies. In some cases, fully human antibodies can be generated from recombinant human antibody library screening techniques as described elsewhere (Griffiths et al., EMBO J., 13:3245-3260 (1994); and Knappik et al., J. Mol. Biol., 296:57-86 (2000)).

Antibodies provided herein can be derived from human antibody fragments isolated from a combinatorial immunoglobulin library. See, for example, Barbas et al., METHODS: A COMPANION TO METHODS IN ENZYMOLOGY, VOL. 2, page 119 (1991) and Winter et al., Ann. Rev. Immunol. 12: 433 (1994). Cloning and expression vectors that are useful for producing a human immunoglobulin phage library can be obtained, for example, from STRATAGENE Cloning Systems (La Jolla, CA).

In addition, antibodies provided herein can be derived from a human monoclonal antibody. Such antibodies can be obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens and can be used to produce human antibody secreting hybridomas. Methods for obtaining human antibodies from transgenic mice are described by Green et al. (Nature Genet., 7:13 (1994)), Lonberg et al. (Nature, 368:856 (1994)), and Taylor et al. (Int. Immunol., 6:579 (1994)).

The antibodies provided herein can be substantially pure. The term “substantially pure” as used herein with reference to an antibody means the antibody is substantially free of other polypeptides, lipids, carbohydrates, and nucleic acid with which it is naturally associated. Thus, a substantially pure antibody is any antibody that is removed from its natural environment and is at least 60 percent pure. A substantially pure antibody can be at least about 65, 70, 75, 80, 85, 90, 95, or 99 percent pure.

A further aspect of the present disclosure is a method of treating a viral infection in a subject having or suspected of having a viral infection. The method includes administering to the subject a pharmaceutical composition, the pharmaceutical composition including a monovalent anti-CD3 antibody, wherein the monovalent anti-CD3 antibody specifically binds to CD3, induces a conformational change in a CD3 complex (CD3Δc), does not initiate CD3 signaling, does not block interaction of a T cell receptor with a viral antigen, and does not block a T cell’s signaling response to the viral antigen; and at least one of the viral antigen and a nucleic acid that expresses the viral antigen.

In another aspect, the present disclosure is directed to a method of increasing an immune response to a viral antigen in a subject in need thereof. The method includes administering to the subject a pharmaceutical composition, the pharmaceutical composition including a monovalent anti-CD3 antibody, wherein the monovalent anti-CD3 antibody specifically binds to CD3, induces a conformational change in a CD3 complex (CD3Δc), does not initiate CD3 signaling, does not block interaction of a T cell receptor with the viral antigen, and does not block a T cell’s signaling response to the viral antigen; and at least one of the viral antigen and a nucleic acid that expresses the viral antigen.

The method can further include analyzing the immune response by the subject as compared to an immune response produced against the viral antigen when the viral antigen or the nucleic acid is administered to a subject in the absence of administration of the pharmaceutical composition. The immune response can comprise the subject producing at least one T cell clone with a CDR3 amino acid sequence selected from the group consisting of CASSKVTGTGNYGYTF (SEQ ID NO: 2), CASSLALNTEAFF (SEQ ID NO: 3), CASSPSTGTIYGYTF (SEQ ID NO: 4), CASSPITGQGAYGYTF (SEQ ID NO: 5), CATFEEPNEKLFF (SEQ ID NO: 6), CASSPWAYATDTQYF (SEQ ID NO: 7), CASSYADRGAGELFF (SEQ ID NO: 8), CASRDRENTEAFF (SEQ ID NO: 9), CASSIDSPNTEAFF (SEQ ID NO: 10), CARTGYEDTEAFF (SEQ ID NO: 11), CASSRTSINEQFF (SEQ ID NO: 12), CASSPEGGGGAFF (SEQ ID NO: 13), CASSPTTGTGTYGYTF (SEQ ID NO: 14), CASSLEGYTEAFF (SEQ ID NO: 15), CASSPIAGYPHEQYF (SEQ ID NO: 16), CASSPGTYGYTF (SEQ ID NO: 17), CASSIMNEQFF (SEQ ID NO: 18), CASSLAPPYEQYF (SEQ ID NO: 19), CASSQGVGLGEKLFF (SEQ ID NO: 20), CASSPRDNPNYGYTF (SEQ ID NO: 21), CASSSVNEQFF (SEQ ID NO: 22), CASSPKTGATYGYTF (SEQ ID NO: 23), CASTPQTGTGYYGYTF (SEQ ID NO: 24), CASGLGVNTEAFF (SEQ ID NO: 25), CASRDGGYEQYF (SEQ ID NO: 26), CASSSRTSGRFYEQYF (SEQ ID NO: 27), CASSLDPSGRLGDEQYF (SEQ ID NO: 28), CASSINYSNQPQHF (SEQ ID NO: 29), CASSPKTGTGTYGYTF (SEQ ID NO: 30), CSANQGGGNTEAFF (SEQ ID NO: 31), CSVTLPQADGRYGYTF (SEQ ID NO: 32), CASSSAYYGYTF (SEQ ID NO: 33), CASSNPGGSSYYEQYF (SEQ ID NO: 34), CASSQEPGNYGYTF (SEQ ID NO: 35), CASSQTRGAGNTIYF (SEQ ID NO: 36), CASSLGQAYEQYF (SEQ ID NO: 37), CASRQGFPGNEQFF (SEQ ID NO: 38), CASRSLRDLNTEAFF (SEQ ID NO: 39), CASSQVPDSDCNQPQHF (SEQ ID NO: 40), CASSEEWGTSGGANEQFF (SEQ ID NO: 41), CASCSTTGYETQYF (SEQ ID NO: 42), CASSLAETENTEAFF (SEQ ID NO: 43), CASSSRFGTGTHEQYF (SEQ ID NO: 44), CASSQDYPPAGGTNNEQFF (SEQ ID NO: 45), CSVEDEDSRTDTQYF (SEQ ID NO: 46), CSAGRGIKTGRSETQYF (SEQ ID NO: 47), CASSRQRTYTGELFF (SEQ ID NO: 48), CASSVAGGLQETQYF (SEQ ID NO: 49), CASSLVGVEAFF (SEQ ID NO: 50), CASSLQTGVAFF (SEQ ID NO: 51), and combinations thereof.

As described herein, monovalent anti-CD3 antibodies provided herein can be used to increase the immune response produced against a viral antigen. Examples of such antigens include viral antigens derived from genetic mutations and atypical gene products, and viral polypeptides. Examples of viruses include human cytomegalovirus (HCMV) and other viruses that can be driven to latency or produce chronic infections. Antigens (e.g., viral antigens) can be administered as, for example, polypeptides (e.g., short or truncated polypeptides or full length polypeptides), DNA encoding such polypeptides, viral particles designed to express such polypeptides, or dendritic cells loaded with such polypeptides.

As used herein, the terms “treating,” “treat,” or “treatment,” refer to restraining, slowing, lessening, reducing, or reversing the progression or severity of an existing symptom, disorder, condition, or disease, or ameliorating clinical symptoms and/or signs of a condition. Beneficial or desired clinical results include alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Those in need of treatment include those already with the disease.

The pharmaceutical compositions described herein may be administered by parenteral routes (e.g., subcutaneous, intravenous, intraperitoneal, intramuscular, or transdermal). Pharmaceutical compositions comprising an antibody for use in the methods of the present invention can be prepared by methods well known in the art (e.g., Remington: The Science and Practice a/Pharmacy, 19th edition (1995), (A. Gennaro et al., Mack Publishing Co.) and comprise an antibody as disclosed herein, a viral antigen, and one or more pharmaceutically acceptable carriers, diluents, or excipients. The pharmaceutical composition may be formulated in a therapeutically effective amount in any conventional dosage forms appropriate for the methods described herein.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

The dosage of each component of the claimed pharmaceutical compositionss depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

As used herein, “subject in need thereof” (also used interchangeably herein with “a patient in need thereof”) refers to a subject susceptible to or at risk of a specified disease, disorder, or condition. The methods disclosed herein can be used with a subset of subjects who have a viral infection. Because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions. Formulations of the present disclosure can be administered to “a subject in need thereof”. As used herein, “a subject” (also interchangeably referred to as “an individual” and “a patient”) refers to animals including humans and non-human animals. Accordingly, the compositions and methods disclosed herein can be used for human and veterinary medical applications. Suitable subjects include warm-blooded mammalian hosts, including humans, companion animals (e.g., dogs, cats), cows, horses, mice, rats, rabbits, primates, and pigs.

The term “administering” as used herein includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and vehicles. Illustrative formats for oral administration include tablets, capsules, elixirs, syrups, and the like. Illustrative routes for parenteral administration include intravenous, intraarterial, intraperitoneal, epidurial, intraurethral, intrasternal, intramuscular and subcutaneous, as well as any other art recognized route of parenteral administration.

Depending upon the disease as described herein, the route of administration and/or whether the compounds and/or compositions are administered locally or systemically with a wide range of permissible dosages. The dosages may be single or divided, and may be administered according to a wide variety of protocols, including q.d., b.i.d., t.i.d., or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

As used in this application, including the appended claims, the singular forms “a,” “an,” and “the” include plural references, unless the content clearly dictates otherwise, and are used interchangeably with “at least one” and “one or more.”

The invention will be further described in the following examples, which do not limit the scope of the invention described in the claims.

### EXAMPLES

### Example 1 - Materials and Methods

The following materials and methods were used throughout the rest of the examples.

### Cell Lines

Previously reported OT1 ab.muCD8ab.JRT3 (OT-I.JRT3) and T2-Kb cells tested negative for mycoplasma and were grown in RPMI (Life Technologies), 10% CosmicCalf serum (HyClone), 2 mM L-glutamine, and penicillin (100 U/mL)/streptomycin (100 pg/mL) (Life Technologies) at 37° C., 5% CO₂.

### PBMCs

With Mayo Institutional Review Board approval, whole human blood was collected from healthy volunteers. Peripheral blood mononuclear cells (PBMCs) were isolated by Ficoll gradient and used fresh or cryopreserved in fetal bovine serum, 10% dimethyl sulfoxide. Where indicated, T cells were isolated untouched using the magnetic human pan, CD4, or CD8-T-cell isolation kits following the manufacturer’s protocols (MACS, Miltenyi Biotec).

### Mono-Fab Preparation

Mono-Fabs were prepared as described previously (Nelson et al., 2012, J Biol Chem 287(51):42936-42950). After papain digestion (Sigma-Aldrich), monoclonal antibody (mAb) digests were terminated with iodoacetamide (Sigma-Aldrich) and dialyzed in phosphate-buffered saline (PBS) with periodic buffer exchange. Fc was removed with Protein A Sepharose (GE Healthcare). Following isolation by size exclusion chromatography over 2 tandem Super-dex200 10/300GL columns (GE Life Sciences) on NGC-Quest10 FPLC system (Bio-Rad), mono-Fabs were sterile-filtered in cold PBS + 2 M L-proline to preserve monovalency. Protein concentration was quantified on DeNovixDS-11 Spectrophotometer.

### Peptides and Antibodies

The peptide ovalbumin (pOVA, SIINFEKL, SEQ ID NO: 52) and the peptide FARL (pFARL, SSIEFARL, SEQ ID NO: 53) were purchased from Elim Biopharmaceuticals. NLV peptide (NLVPMVATV, SEQ ID NO: 1) was synthesized by the Mayo Clinic Rochester Proteomics Core. Culture supernatants of OKT3 hybridoma were purified to obtain OKT3 mAb. The following antibodies were purchased: UCHT1 mAb (BioXCell), anti-CD8 DK25 mAb (Agilent), serum mouse immunoglobulin G (Ms-IgG) and Ms-IgG-Fab (Jackson ImmunoResearch), anti-mouse IgG (BioLegend, Poly4060), anti-human CD4 (BD Biosciences, RPA-T4, OKT4), anti-human CD8 (BD Biosciences, HIT8a, RPA-T8, SK1), anti-human CD69 (BD Biosciences, FN50), anti-human CD45 (BioLegend, UCHL1), anti-human CD56 (BD Biosciences, B159), anti-human CD19 (BioLegend, HIB19), anti-V35 (BioLegend, MR9-4), and anti-Nur77 (Invitrogen, 12.14). H2-Kb/OVA-tetramer was made as previously described (Johnson et al., 1999, J Virol 73(5):3702-3708). HLA-A*02:01/NLV (A2/NLV)-tetramer was purchased from MBL International. GhostDye discriminated live/dead cells (Tonbo).

### Flow Cytometry

Cells stained with indicated fluorophore-conjugated antibodies were collected on either Guava easyCyte HT Flow Cytometer (Luminex) or BD-Accuri C6 Flow Cytometer (BD Biosciences). For intracellular staining of Nur77, samples were fixed and permeabilized with Cytofix/Cytoperm Kit per the manufacturer’s protocol (BD Biosciences). Data analysis was performed using FlowJo (Tree Star) or guavaSoft software.

### T-Cell Activation

A total of 50,000 OT-I.JRT3 T cells per well were stimulated with 0.2 nM indicated peptides presented by T2-Kb APCs and 10 pg/mL Ms-IgG-Fab control or specific mono-Fabs for 24 hours. Human PBMCs were rested overnight following fresh isolation or thaw from cryopreservation. Next, 0.2 × 10⁶ PBMCs per well were stimulated with indicated control or specific immunoglobulins (10 pg/mL) for 4 to 6 hours.

### Western Blot

A total of 1 × 10⁶ human PBMCs were stimulated with 10 pg/mL immunoglobulins as indicated in the presence or absence of pervanadate for 5 minutes, 37° C. Cells were washed twice in cold PBS and lysed for 10 minutes in 1% TritonX-100, 20 mM Tris/HCl pH 7.4, and 150 mM NaCl plus Halt-protease/phosphatase inhibitors (ThermoFisher). Equivalent cell lysates were subjected to SDS-PAGE (reducing, 10% gel), polyvinylidene difluoride membrane transfer, and western blot analysis with anti-phosphotyrosine (EMD Millipore, 4G10) and secondary anti-mouse IgG horseradish peroxidase (Cell Signaling).

### CD3 Pull-Down (CD3-PD)

The CD3-PD assay was used to quantify CD3 conformational change (CD3ic) by detection of CD3e proline-rich sequence exposure. 30 × 10⁶ PBMCs per sample were lysed in isotonic ice-cold buffer containing 1% Brij 58 (Sigma-Aldrich) and centrifuged to obtain postnuclear fractions. Samples were precleared with glutathione S-transferase (GST) beads (4° C., 1 hour) in the presence of indicated immunoglobulins (10 pg/mL), followed by specific CD3-PD with GST-SH3.1-NCK beads (4° C., 12 hours). CD3-PD samples were subjected to SDS-PAGE (reducing, 13% gel), nitrocellulose transfer, and western blot with rabbit serum 448 antibody, specific for CD3 (from Balbino Alarcón, Universidad Autónoma de Madrid, Madrid, Spain). The mAb APA 1/1 (GE Biosciences) set the assay background level. Protein acetone precipitates from a fraction of postnuclear lysates controlled for total CD3 content per sample. Quantification was performed as described previously (de la Cruz et al., 2011, J Immunol 186(4):2282-2290).

### Recall T-Cell Expansion Cultures

CD8 T cells were expanded as described previously (Montes et al., 2005, Clin Exp Immunol 142(2):292-302), with minor modifications. 0.2 × 10⁶ total PBMCs per well were seeded on day 0 with no exogenous peptide or 1 pM exogenous NLV peptide in RPMI, 10% fetal bovine serum. On day 2, 10 pg/mL mono-Fab and 20 U/mL interleukin-2 (IL-2) (Proleukin, Mayo Pharmacy) were added to culture. On days 4 and 7, half the media was replaced with fresh media containing 20 U/mL IL-2. Flow cytometry was run on day 9. For antigen-blocking experiments, 5 pg/mL DK25 antibody or Ms-IgG control was added on days 0, 2, and 4, and flow cytometry was run on day 7.

### ELISA

Supernatants were collected from day 7 recall assays and stored at -20° C. Supernatants were thawed at room temperature and analyzed for human interferon-γ (IFN-γ) and granzyme B levels by sandwich enzyme-linked immunosorbent assay (ELISA) according to manufacturer’s protocol (R&D Systems).

### Cytotoxic T Lymphocyte (CTL) Assay

Performed as described previously (Noto et al., 2013, J Vix Exp 82:e51105), CD8 T cells from NLV-expanded recall assays and target CD4 T cells from thawed, autologous PBMCs were isolated using negative-selection magnetic beads. CD4 T cells were loaded with 10 pM NLV and labeled with 0.01 pM carboxyfluorescein diacetate succinimidyl ester (specific targets) or not loaded but labeled with 0.1 pM carboxyfluorescein diacetate succinimidyl ester (nonspecific targets). Specific and nonspecific targets were mixed 1:1 for coculture with serially diluted recall CD8 T cells overnight and then analyzed by flow cytometry. Specific lysis was calculated based on the ratio of live NLV-loaded/nonloaded target cells.

### TRBV-CDR3 Sequencing and Analysis

Genomic DNA was isolated from frozen cell pellets (QIAamp DNA mini kit). TRBV-CDR3 sequencing and preliminary analysis was completed using the immunoSEQ platform (Adaptive Biotechnologies, hsTCR3 kit). Per the manufacturer’s protocol, 1.6 pg genomic DNA per sample was subjected to polymerase chain reaction (PCR) to amplify all TRBV-CDR3 sequences in a bias-controlled manner using multiplexed V- and J-gene primers. Amplified TRBV-CDR3 underwent a second PCR to generate barcoded libraries. Sequencer-ready barcoded libraries were pooled and sequenced on an Illumina MiSeq. Raw sequencing data were sent to Adaptive Biotechnologies for processing to report the normalized, annotated TCR-3 repertoire of each sample. Data analysis was performed using the provided immunoSEQ Analyzer program. The VDJdb database was accessed to identify TRBV-CDR3 clones in the data set matching those from previously published public TCRs associated with HLA-A2 and NLV peptide. “Public” TCR was operationally defined as 100% identity of TRBV-CDR3 amino acid sequence between individuals; it is possible that clones classified as private here could be found public upon deeper/broader population sequencing.

### Statistical Analysis

Statistics performed using GraphPad Prism included 2-tailed, 1-tailed, unpaired, and paired Student t tests and Fisher’s exact tests. Results showing central values represent mean ± standard deviation (SD) or standard error of the mean (SEM).

### Example 2 - Mono-OKT3-Fab Binds to Human CD3 Without Blocking TCR-Antigen Interactions

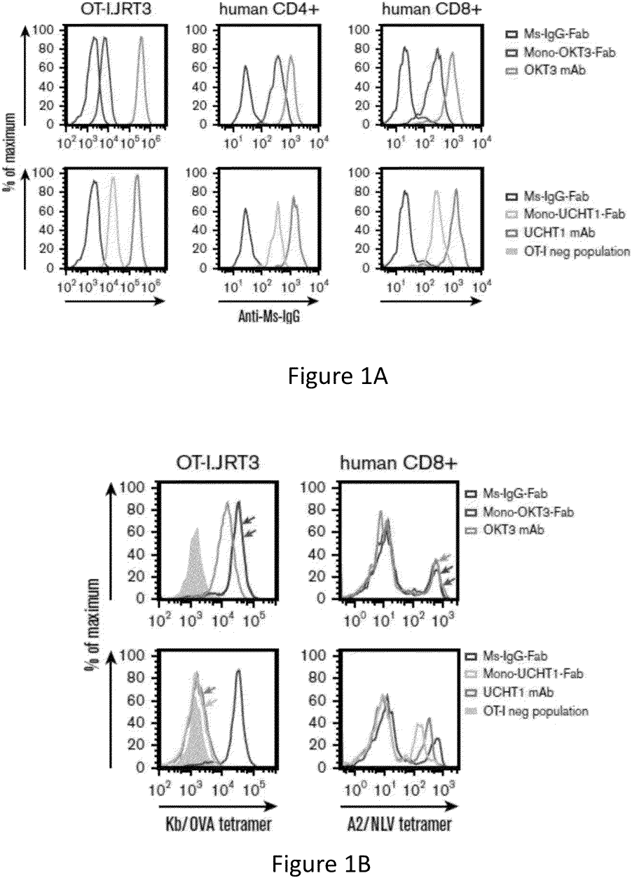

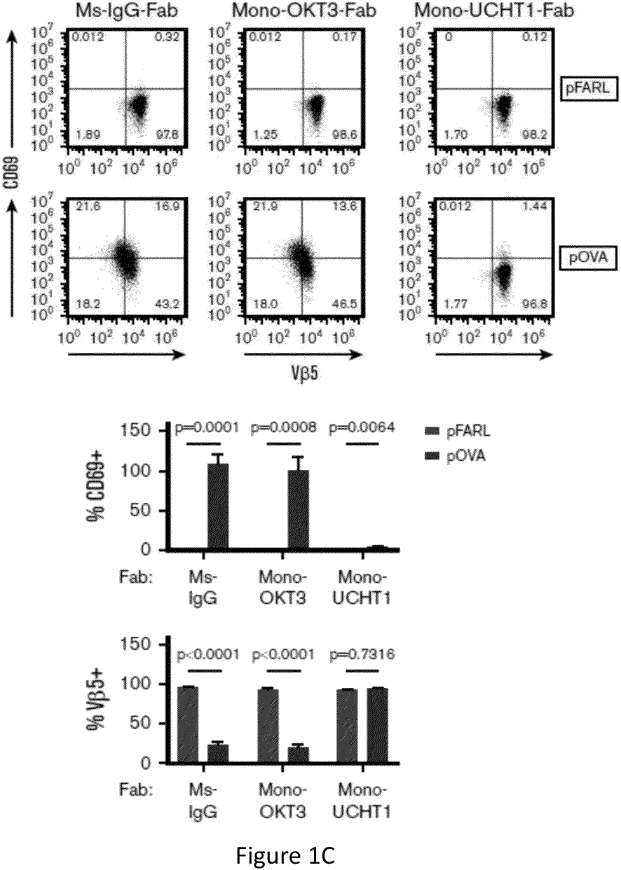

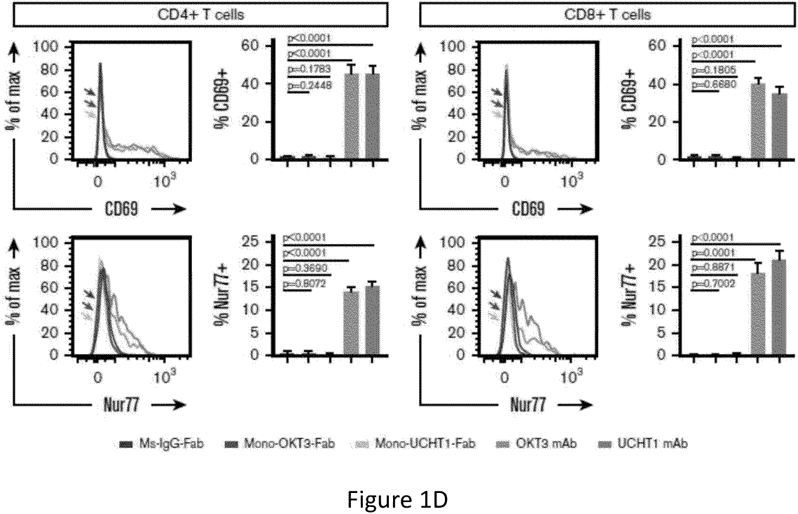

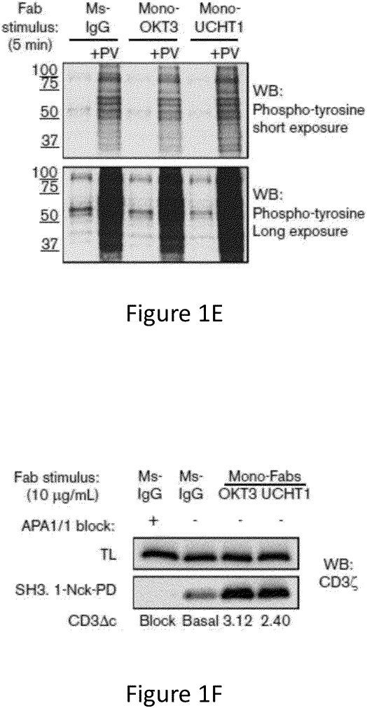

Binding distinct but overlapping epitopes of human CD3ε, mAbs OKT3 and UCHT1 were subjected to papain digestion to obtain mono-Fabs. For copotentiation, mono-Fabs must bind CD3 without sterically hindering TCR-antigen binding or signaling. OKT3 and UCHT1 mono-Fabs bound surface CD3 of OT-I.JRT3 cells, expressing mouse CD8a3 and OT-I-TCR-a3 in complex with human CD3 (FIG. 1A, left). Both mono-Fabs also bound primary human CD4 and CD8 T cells (FIG. 1A, middle and right). When bound, mono-OKT3-Fab did not block TCR-antigen interaction, as demonstrated by unaffected Kb/OVA-tetramer staining of OT-I.JRT3 cells (FIG. 1B, top left) and uninterrupted A2/NLV-tetramer staining of HLA-A*02:01 (+) CD8 T cells (FIG. 1B, top right). Furthermore, binding of mono-OKT3-Fab to OT-I.JRT3 cells did not alter surface TCR downregulation or CD69 upregulation in response to SIINFEKL antigenic peptide (FIG. 1C, pOVA). In contrast, mono-UCHT1-Fab inhibited TCR-antigen interaction in both OT-I.JRT3 and human A2/NLV-tetramer(+) CD8 T cells (FIG. 1B, bottom) and inhibited surface TCR downregulation and CD69 upregulation of OT-I.JRT3 cells in response to SIINFEKL (FIG. 1C, pOVA).

### Example 3 - Mono-OKT3-Fab Induces CD3Δc Without Initiating Early Signaling

In the absence of antigen recognition, neither mono-Fab induced signaling-dependent surface TCR downregulation or CD69 upregulation in OT-I.JRT3 cells, as expected for noncrosslinking species (FIG. 1C, pFARL). Likewise, neither mono-Fab induced CD69 or Nur77 upregulation in primary human T cells, unlike their parent bivalent mAbs (FIG. 1D). Furthermore, mono-Fabs did not induce accumulation of tyrosine-phosphorylated proteins following engagement of human PBMCs compared with positive control, pervanadate (FIG. 1E). Despite their inability to trigger CD3 signaling, mono-Fab binding induced a conformational change in CD3 (CD3Δc), as indicated by a CD3-PD assay (FIG. 1F), whereas GST-SH3.1-Nck beads capture TCR/CD3 complexes displaying a CD3ε proline-rich sequence exposed upon optimal TCR engagement. Based on its ability to bind human CD3 and induce CD3ic without blocking TCR-antigen interaction and without intrinsically initiating early signaling, mono-OKT3-Fab was selected to study copotentiation of human T cells.

### Example 4 - Mono-OKT3-Fab Enhances Recall T-Cell Response to Autologous APCs and NLV:HLA-A2

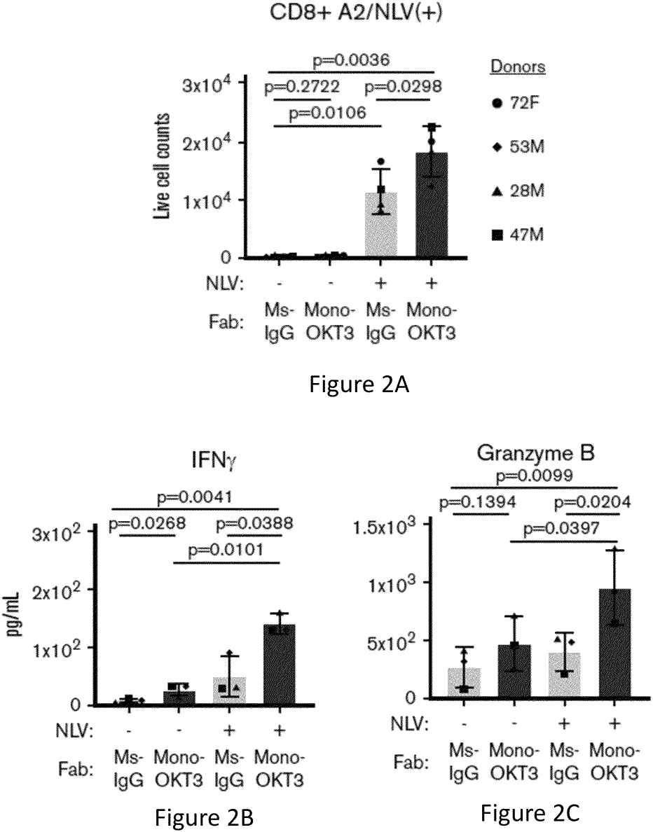

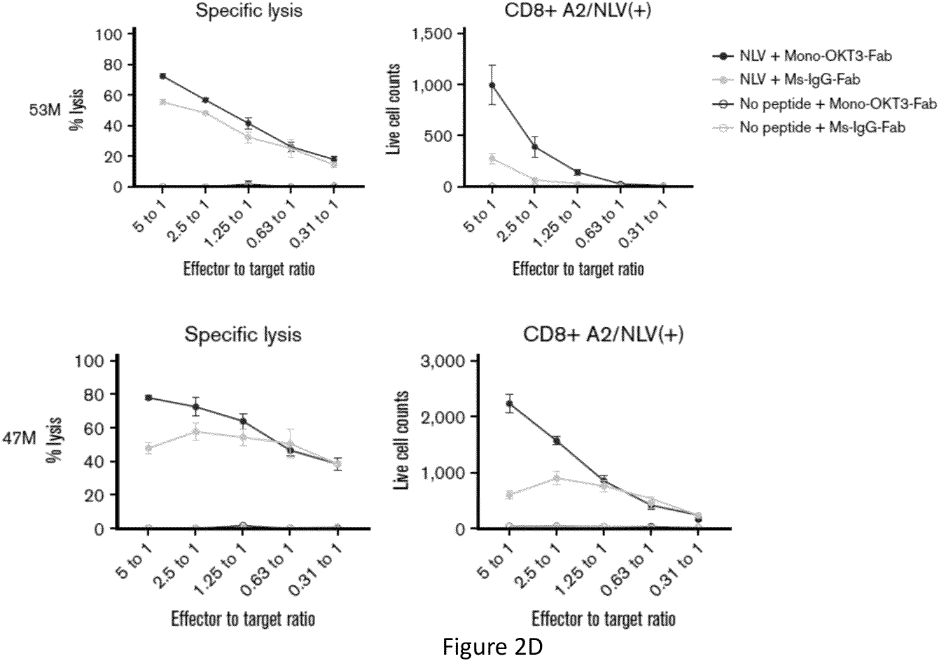

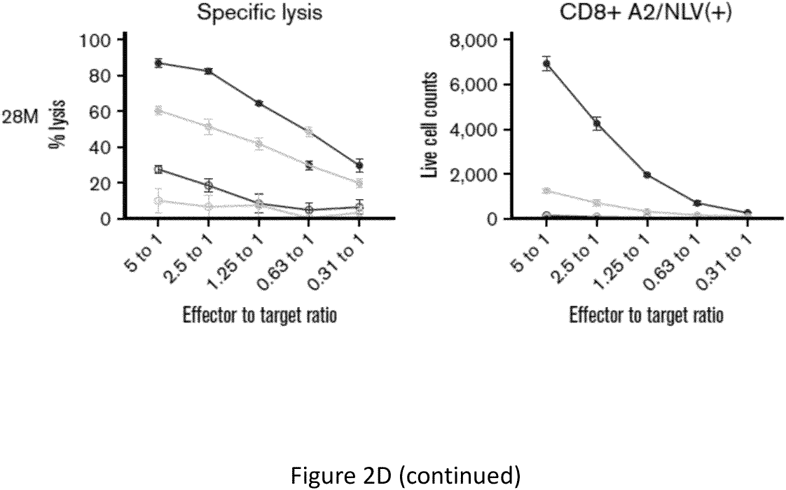

Seven healthy blood donors were classified by HLA-A*02:01 expression and the presence of CD8 T cells positive for binding A2/NLV-tetramer, an immunodominant antigen and marker of HCMV positivity. Four donors were HLA-A*02:01(+) and A2/NLV-tetramer(+) (72F, 53M, 28M, and 47M), 1 donor was HLA-A*02:01(+) but A2/NLV-tetramer(-) (74M), and 2 donors were HLA-A*02:01(-) and A2/NLV-tetramer(-) (78F and 59F). Bulk PBMCs were tested in recall T-cell expansion assays driven by exogenous NLV peptide. On day 0, donors presented variable numbers of bulk CD8 T cells, including low but detectable A2/NLV-tetramer(+) CD8 T cells in the expected prescreened donors. After culturing PBMCs for 9 days in the presence of exogenous NLV + irrelevant Ig + late IL-2, donors positive for A2/NLV tetramer presented higher CD8 T-cell counts than cultures without exogenous peptide (FIG. 2A, NLV: +, Fab: Ms-IgG). These donors were categorized as exogenous NLV responsive in bulk culture (exog-NLV-bulk-responsive). For these donors, NLV + mono-OKT3-Fab increased A2/NLV-tetramer(+) CD8 T-cell numbers even more (FIG. 2A, NLV: +, Fab: Mono-OKT3). NLV + mono-OKT3-Fab stimulation also induced significant increase in IFN-y and granzyme B accumulation by day 7 in culture supernatants (FIGS. 2B-2C). Bulk CD8 T cells isolated at day 7 from NLV recall cultures ± mono-OKT3-Fab were tested for CTL activity against autologous NLV-loaded CD4⁺ target cells. Greater specific lysis was observed in the mono-OKT3-Fab copotentiated cultures (FIG. 2D, left panels) correlating with increased NLV-A2-tetramer(+) cells (FIG. 2D, right panels), demonstrating anti-NLV functional specificity.

### Example 5 - CD3 Copotentiation Is Dependent on TCR-HLA and CD8 Coreceptor Engagement

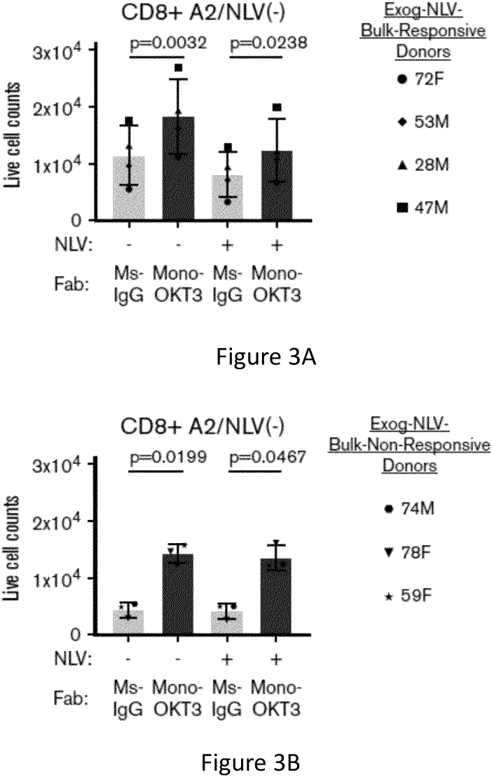

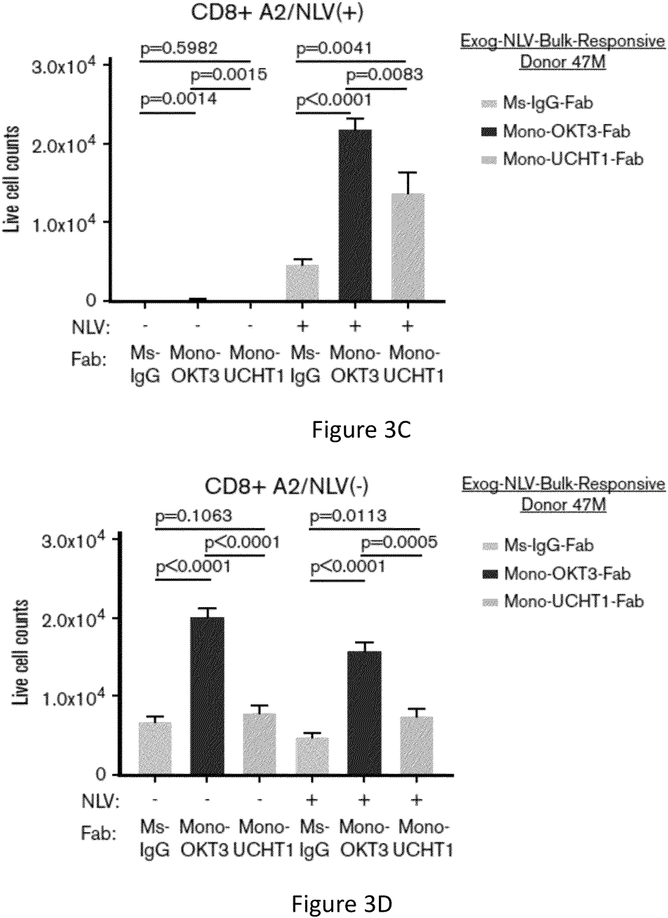

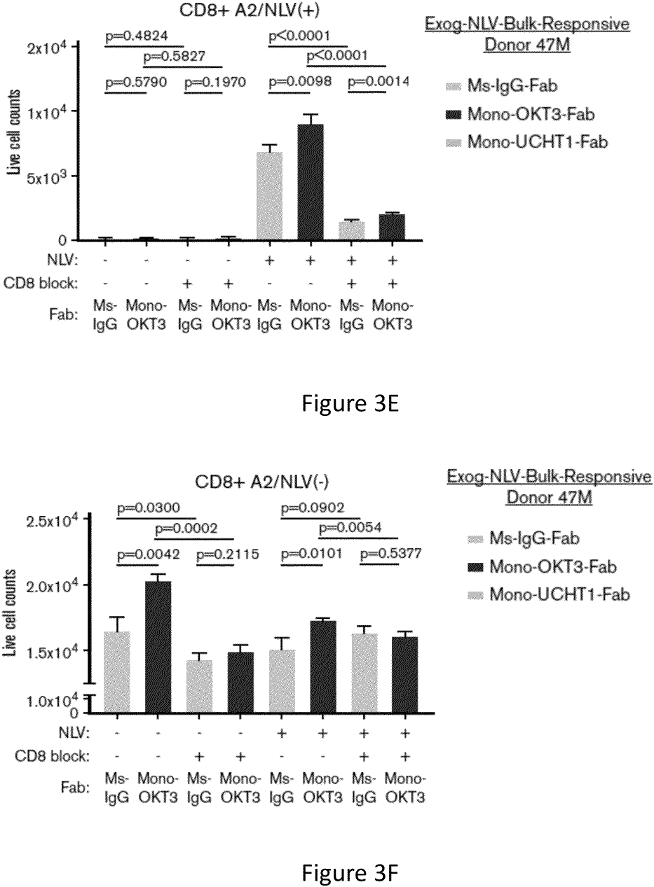

A2/NLV-tetramer(-) CD8 T cells in the same recall cultures increased in the presence of mono-OKT3-Fab, but not exogenous NLV peptide (FIG. 3A), and PBMCs from A2/NLV-tetramer(-) donors responded likewise (FIG. 3B). Thus, mono-OKT3-Fab increased the numbers of both A2/NLV-tetramer(+) and A2/NLV-tetramer(-) CD8 T cells. To distinguish mono-OKT3-Fab intrinsic T-cell stimulation from TCR-HLA-dependent copotentiation, recall assays were performed in the presence or absence of blocking reagents to TCR-HLA or CD8 coreceptor. First, CD8 T cells from 1 exog-NLV-bulk-responsive donor were cultured in the presence of mono-OKT3-Fab or mono-UCHT1-Fab, the latter binding to CD3 and inducing CD3Δc but impairing antigen binding to T cells (FIGS. 1A-1F). Mono-UCHT1-Fab significantly reduced copotentiation when compared with mono-OKT3-Fab of CD8 T cells either positive (FIG. 3C) or negative (FIG. 3D) for A2/NLV-tetramer. These results indicate that with impaired TCR-antigen interactions, induction of CD3Δc by mono-Fabs is insufficient to mediate copotentiation. Second, parallel experiments showed that the anti-CD8 blocking antibody, DK25, inhibited both exogenous NLV-specific and mono-OKT3-Fab-specific responses in CD8 T cells either positive (FIG. 3E) or negative (FIG. 3F) for A2/NLV tetramer. Thus, mono-OKT3-Fab copotentiation is dependent on CD8-TCR-HLA engagement for both A2/NLV-tetramer(+) CD8 T cells driven by exogenous NLV and for A2/NLV-tetramer(-) CD8 T cells driven only by autologous APCs.

### Example 6 - Mono-OKT3-Fab Copotentiation Primarily Enhances Expansion of Top-Ranked T-Cell Clones

To analyze the T-cell clonal dynamics of the copotentiation response, recall assays were followed by DNA extraction and TRBV-CDR3 analysis via immunoSEQ with single-cell resolution. The number of clones sampled per donor per culture condition reached as high as ~50,000. Clonal diversity was estimated by scaled Shannon entropy, a value whose range is 0 to 1, where 0 represents minimum diversity exhibited by a monoclonal T-cell population and 1 represents maximal repertoire diversity when all TRBV-CDR3 sequences are expressed equally. It was observed that exogenous NLV decreased entropy compared with negative-control cultures for 4 out of 4 exog-NLV-bulk-responsive donors and likewise decreased the total clone number sampled from cultures, an expected outcome when T-cell clones specific for a single peptide proliferate and increase their relative representation. In contrast, mono-OKT3-Fab did not reliably produce such an effect (observed in 2/4 exog-NLV-bulk-responsive donors), nor did mono-OKT3-Fab tend to further decrease entropy when administered in combination with exogenous NLV compared with NLV alone. Thus, mono-OKT3-Fab was not producing a clonal effect identical to that of exogenous peptide.

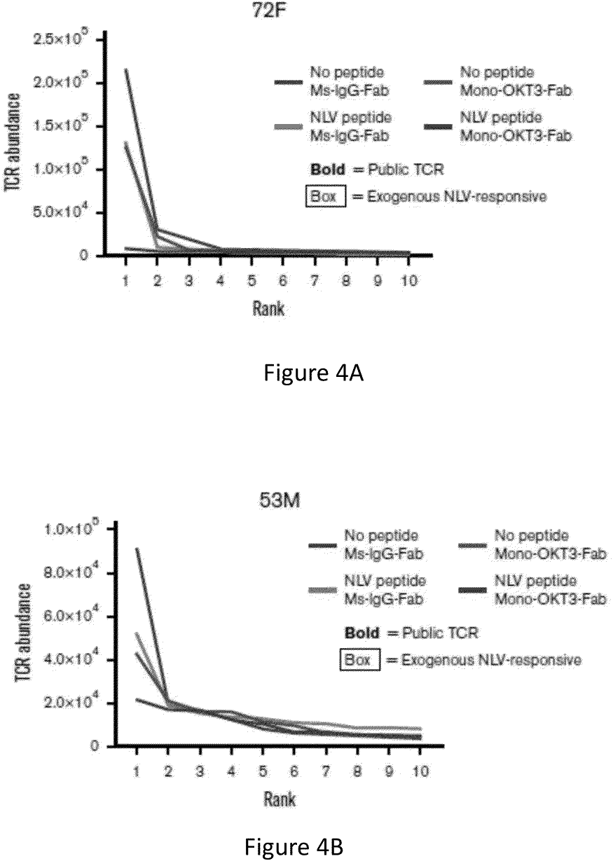

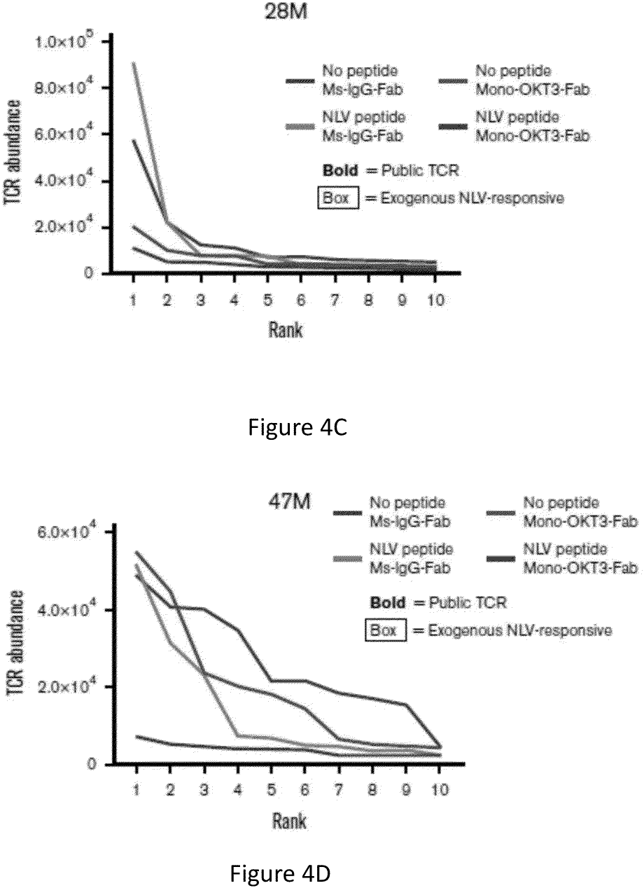

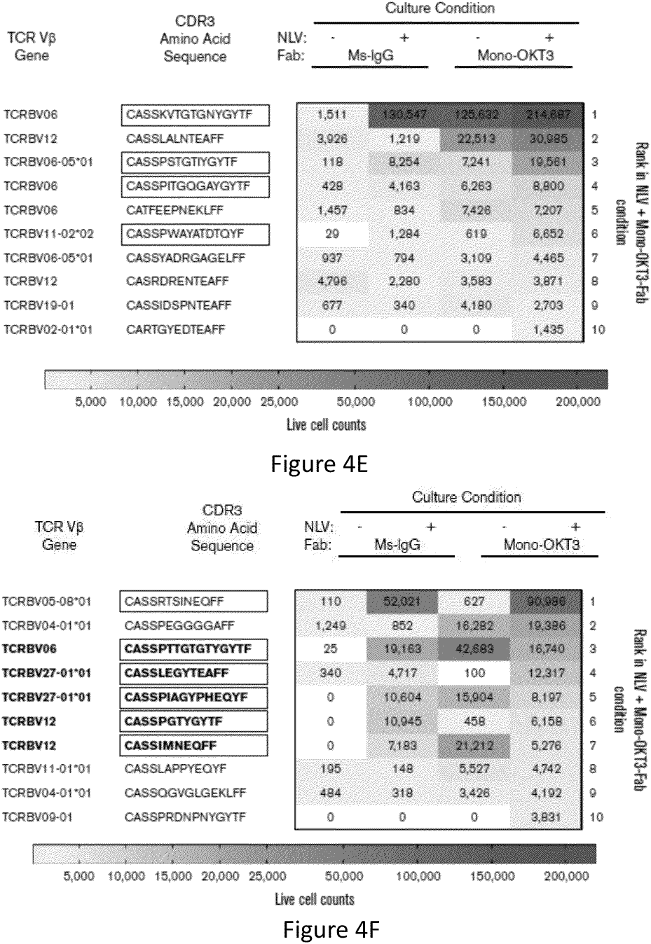

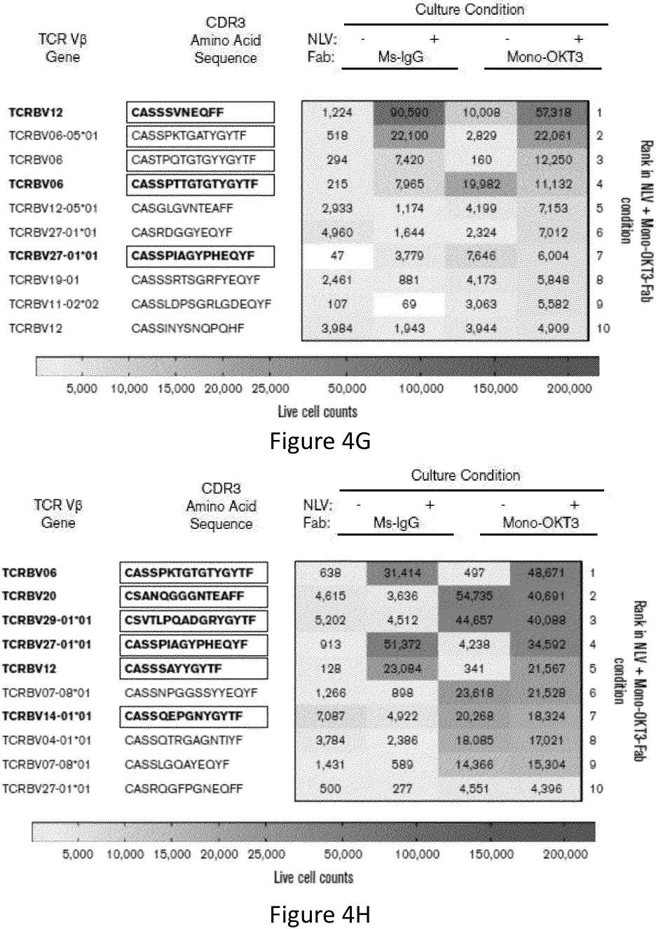

To determine if mono-OKT3-Fab indiscriminately caused many clones to expand, single-cell clonal sequencing data was used to estimate total copy number of each clone in recall cultures and compared between conditions by rank analysis. It was found that despite thousands of clones measured, substantial differences in cell numbers ranked according to abundance were heavily concentrated in the top 10 clones (FIGS. 4A-D). Thus, copotentiation must have a mechanism of clonal specificity, which is more deeply analyzed here, discussing the HLA-A2(+) exog-NLV-bulk-responsive donor 72F as an example. Among the top 10 clones from negative-control cultures, none were NLV responsive, and most scored as “zeros” in the other culture conditions. This likely indicates the individual clones were too infrequent for consistent sampling. In exogenous NLV cultures, the top clone reached ~130,000 cells, having represented ~1500 cells in negative-control culture. Several other top clones were absent in ≥1 other culture conditions, and thus, as above, some analysis had sampling limitations. However, ranks 3 and 5 were consistently sampled and showed matching clones that were also amplified in the mono-OKT3-Fab-only condition, with synergistic highest abundance in NLV + mono-OKT3-Fab cultures. Rank 8 was already high in negative-control culture and was neither exogenous NLV-responsive nor amplified by mono-OKT3-Fab. In mono-OKT3-Fab-only cultures, 3 out of 10 top clones were independently NLV responsive in exogenous NLV cultures, while 4 out of 10 top clones responded to mono-OKT3-Fab, but not exogenous NLV. Finally, in NLV + mono-OKT3-Fab cultures (FIG. 4E), 4 out of 10 top clones were NLV responsive and synergistically amplified, while 5 out of 10 top clones were not NLV responsive but were amplified to a similar extent as mono-OKT3-Fab-only cultures. Therefore, copotentiation amplified clonal abundance of certain top clones only, with some, but not others, also being responsive to exogenous NLV.

### Example 7 - Different Classes of T-Cell Clones Respond to CD3 Copotentiation With Distinct Clonal Expansion Signatures

The other 3 exog-NLV-bulk-responsive donors showed similar examples of clonal responses (FIGS. 4F-H), while unlike donor 72F, the others had exogenous NLV-responsive public TCRs among top clones, which in 12 out of 14 occurrences responded to mono-OKT3-Fab. However, there was an unexpected pattern in their response: public clones tended to respond best to either mono-OKT3-Fab only or exogenous NLV but less optimally to the combination. Assigning first-place performance (“gold”) to conditions with highest clonal abundance and second/third-place (“silver-bronze”), it was found that considering all top 10 clones, private much more than public TCRs showed synergy in combination treatment (Table 1, shown below).

In Table 1, TCR clones ranked in the top 10 from the LNV + mono-OKT3-Fab condition with evidence of NLV specificity were analyzed according to their abundance in various recall culture conditions. For each TRBV-CDR3 amino acid sequence, gold was awarded to conditions with the highest clonal abundance, silver to conditions with the second highest clonal abundance, and bronze to conditions with the third highest clonal abundance. Bolded sequences indicate public TCRs, while the others are private TCRs. Evidence for NLV specificity was accepted as displaying higher clone numbers in NLV + Ms-IgG-Fab versus no exogenous peptide + Ms-IgG-Fab conditions or, for public TCRs, that pattern in ≥1 other donor or previously reported in the literature. The tendency for public TCRs to score gold in NLV-only or mono-OKT3-Fab-only treatments and private TCRs to score gold in combination treatment was statistically significant (P = 0.003, 2-tailed Fisher’s exact test; P = 0.007, χ² test with Yates correction).

TABLE 1

| Exog-NLV-bulk-responsive donors | | | | Exog-NLV + Ms-IgG Fab | | | No Exog-NLV + mono-OKT3 Fab | | | Exog-NLV + mono-OKT3 Fab | | |
|---|---|---|---|---|---|---|---|---|---|---|---|---|
| Donor | TCR Vβ gene | CDR3 amino acid sequence | SEQ ID NO | TCR abundance | Rank | Award | TCR abundance | Rank | Award | TCR abundance | Rank | Award |
| 72F | TCRBV06 | CASSKVTGTGNYGYTF | 2 | 130547 | 1 | Silver | 125632 | 1 | Bronze | 214687 | 1 | Gold |
| | TCRBV06-05*01 | CASSPSTGTIYGYTF | 4 | 8254 | 3 | Silver | 7241 | 4 | Bronze | 19561 | 3 | Gold |
| | TCRBV06 | CASSPITGQGAYGYTF | 5 | 4163 | 5 | Bronze | 6263 | 5 | Silver | 8800 | 4 | Gold |
| | TCRBV11-02*02 | CASSPWAYATDTQYF | 7 | 1284 | 15 | Silver | 619 | 39 | Bronze | 6652 | 6 | Gold |
| 53M | TCRBV05-08*01 | CASSRTSINEQFF | 12 | 52021 | 1 | Silver | 627 | 67 | Bronze | 90986 | 1 | Gold |
| | TCRBV27-01*01 | CASSLEGYTEAFF | 15 | 4717 | 18 | Silver | 100 | 119 | Bronze | 12317 | 4 | Gold |
| 28M | TCRBV06 | CASTPQTGTGYYGYTF | 24 | 7420 | 5 | Silver | 160 | 86 | Bronze | 12250 | 3 | Gold |
| 47M | TCRBV06 | CASSPTTGTGTYGYTF | 14 | 31414 | 2 | Silver | 497 | 47 | Bronze | 48671 | 1 | Gold |
| 53M | TCRBV06 | CASSPTTGTGTYGYTF | 14 | 19163 | 2 | Silver | 42683 | 1 | Gold | 16740 | 3 | Bronze |
| | TCRBV27-01*01 | CASSPIAGYPHEQYF | 16 | 10604 | 7 | Silver | 15902 | 4 | Gold | 8197 | 5 | Bronze |
| | TCRBV12 | CASSPGTYGYTF | 17 | 10945 | 6 | Gold | 458 | 84 | Bronze | 6158 | 6 | Silver |
| | TCRBV12 | CASSIMNEQFF | 18 | 7183 | 12 | Silver | 21212 | 2 | Gold | 5276 | 7 | Bronze |
| 28M | TCRBV12 | CASSSVNEQFF | 22 | 90590 | 1 | Gold | 10008 | 2 | Bronze | 57318 | 1 | Silver |
| | TCRBV06-05*01 | CASSPKTGATYGYTF | 23 | 22100 | 2 | Gold | 2829 | 12 | Bronze | 22061 | 2 | Silver |
| | TCRBV06 | CASSPTTGTGTYGYTF | 14 | 7965 | 3 | Bronze | 19982 | 1 | Gold | 11132 | 4 | Silver |
| | TCRBV27-01*01 | CASSPIAGYPHEQYF | 16 | 3779 | 6 | Bronze | 7646 | 3 | Gold | 6004 | 7 | Silver |
| 47M | TCRBV20 | CSANQGGGNTEAFF | 31 | 3636 | 8 | Bronze | 54735 | 1 | Gold | 40691 | 2 | Silver |
| | TCRBV29-01*01 | CSVTLPQADGRYGYTF | 32 | 4512 | 7 | Bronze | 44657 | 2 | Gold | 40088 | 3 | Silver |
| | TCRBV27-01*01 | CASSPIAGYPHEQYF | 16 | 51372 | 1 | Gold | 4238 | 10 | Bronze | 34592 | 4 | Silver |
| | TCRBV12 | CASSSAYYGYTF | 33 | 23084 | 3 | Gold | 341 | 63 | Bronze | 21567 | 5 | Silver |
| | TCRBV14-01*01 | CASSQEPGNYGYTF | 35 | 4922 | 6 | Silver | 20268 | 4 | Gold | 18324 | 7 | Silver |

The T-cell clonal dynamics of copotentiation in the A2/NLV-tetramer(-) donors were next examined, all of which amplified T-cell expansion by mono-OKT3-Fab but were exog-NLV-bulk-nonresponsive (FIG. 3B). Exogenous NLV and mono-OKT3-Fab status did not correlate with predictable changes in entropy and total clones sampled, although rank differences remained largely concentrated in top-10 clones. The top clone for each donor appeared exogenous NLV responsive, as did several other top clones, with further enhancement combined with mono-OKT3-Fab. To assess NLV + mono-OKT3-Fab combinatorial synergy, a gold/silver-bronze analysis was applied to these 13 apparently NLV-responsive clones and found that the NLV + mono-OKT3-Fab condition produced the highest clonal abundance in all cases (Table 2, shown below).

Table 2 shows gold/silver-bronze analysis applied to exog-NLV-bulk-nonresponsive donors for the top clones showing greater abundance in NLV + Ms-IgG-Fab versus no peptide + Ms-IgG-Fab conditions. It was observed that for each of these clones, the NLV + mono-OKT3-Fab combination condition yielded greatest abundance.

TABLE 2

| Exog-NLV-bulk-nonresponsive Donors | | | | | TCR Abundance | Rank | TCR Abundance | Rank | TCR Abundance | Rank |
|---|---|---|---|---|---|---|---|---|---|---|
| Donor | TCR Vβ Gene | SEQ ID NO | CDR3 Amino Acid Sequence | Exog NLV: | + | | - | | + | |
| | | | | Fab: | Ms-IgG | | Mono-OKT3 | | Mono-OKT3 | |
| 74 M | TCRBV12 | 39 | CASRSLRDLNTEAFF | | 774 | 22 | 13,325 | 2 | 32,446 | 1 |
| | TCRBV03 | 40 | CASSQVPDSDCNQPQHF | | 132 | 73 | 1,093 | 18 | 2,797 | 6 |
| | TCRBV02-01*01 | 41 | CASSEEWGTSGGANEQFF | | 39 | 97 | 630 | 33 | 1,660 | 8 |
| 78F | TCRBV27-01*01 | 42 | CASCSTTGYETQYF | | 1,467 | 9 | 5,467 | 3 | 23,002 | 1 |
| | TCRBV07-02*01 | 43 | CASSLAETENTEAFF | | 678 | 23 | 2,554 | 11 | 2,762 | 9 |
| 59F | TCRBV07-09 | 44 | CASSSRFGTGTHEQYF | | 4,453 | 3 | 13,023 | 1 | 16,143 | 1 |
| | TCRBV04-03*01 | 45 | CASSQDYPPAGGTNNEQFF | | 431 | 53 | 4,886 | 4 | 11,334 | 2 |
| | TCRBV29-01*01 | 46 | CSVEDEDSRTDTQYF | | 227 | 88 | 2,417 | 7 | 7,824 | 3 |
| | TCRBV20 | 47 | CSAGRGIKTGRSETQYF | | 237 | 86 | 904 | 28 | 5,360 | 4 |
| | TCRBV03 | 48 | CASSRQRTYTGELFF | | 57 | 124 | 1,313 | 21 | 3,537 | 7 |
| | TCRBV06-05*01 | 49 | CASSVAGGLQETQYF | | 66 | 122 | 261 | 75 | 3,050 | 8 |
| | TCRBV11-02*02 | 50 | CASSLVGVEAFF | | 62 | 123 | 1,226 | 24 | 2,987 | 9 |
| | TCRBV07-09 | 51 | CASSLQTGVAFF | | 1,760 | 15 | 1,539 | 16 | 2,274 | 10 |

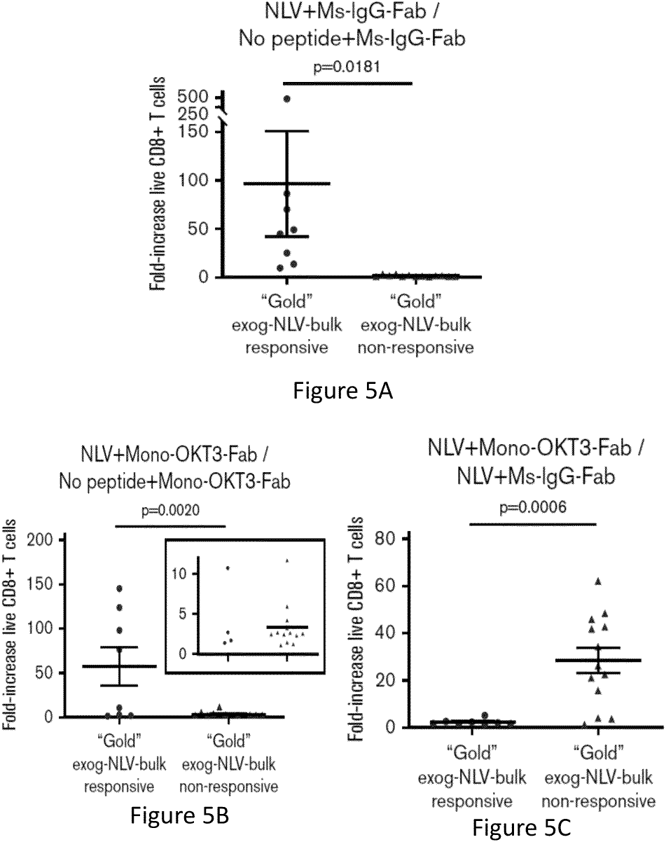

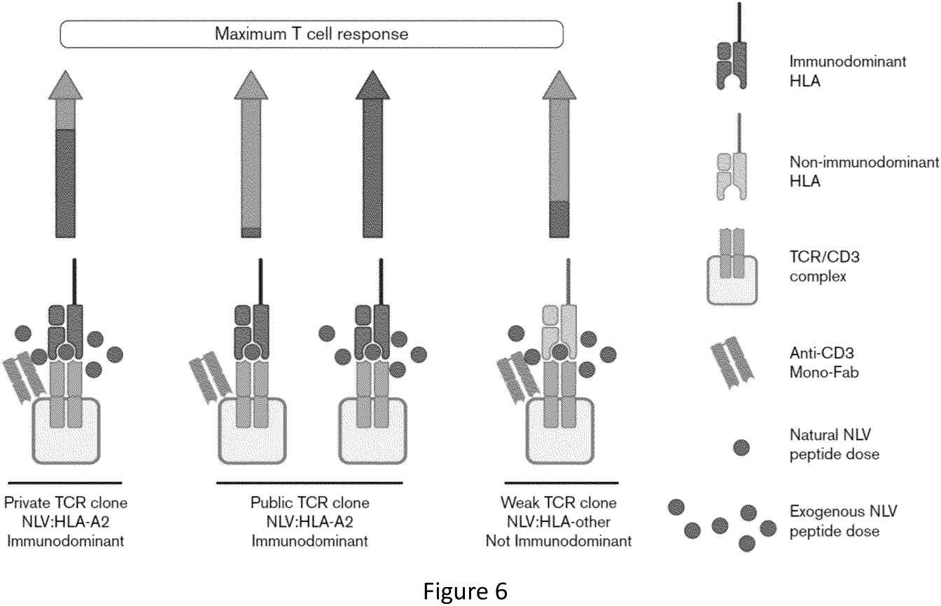

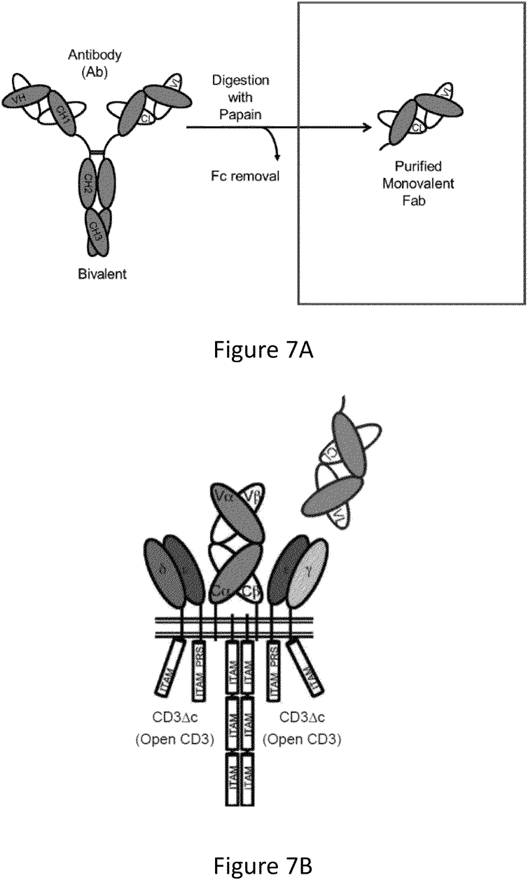

This was similar to the “private TCR” signature noted previously for exog-NLV-bulk-responsive donors, but there was also a distinct difference. The exogenous NLV-only condition increased the cell number of these clones above the negative-control culture condition (~1- to 4-fold); in contrast, combination-treatment “gold-response” clones from exog-NLV-bulk-responsive donors were much more peptide responsive, (~10- to 500-fold; FIG. 5A). Comparing clonal cell numbers from cultures with mono-OKT3-Fab ± exogenous NLV, combination-treatment gold-response clones from exog-NLV-bulk-responsive donors appeared in 2 clusters: one for which exogenous NLV peptide increased clonal abundance by ~75- to 150-fold and another that only increased ~1- to 11-fold; in contrast, NLV-responsive clones from exog-NLV-bulk-nonresponsive donors all appeared in the low-peptide-response cluster (FIG. 5B). This pattern flipped when assessing the contribution of mono-OKT3-Fab to combinatorial synergy; here, exog-NLV-bulk-responsive donor gold-response clones increased ~2-fold on average, while gold-response clones from exog-NLV-bulk-nonresponsive donors increased ~30-fold (FIG. 5C). Taken together, these data show that exog-NLV-bulk-responsive donors responded to CD3 copotentiation by amplifying potent NLV-focused clones, while exog-NLV-bulk-nonresponsive donors responded to combination treatment with synergy driven mostly by CD3 copotentiation and low-but-positive intrinsic potency toward exogenous NLV. Thus, mono-OKT3-Fab provides antigen-specific CD3 copotentiation that can increase expansion of recall public and private clones against antigens that are immunodominant or of intrinsically weak potency (FIG. 6).

### Example 8 - CD3 Copotentiation Mechanisms and Applications

The Examples provided herein demonstrate copotentiation using anti-CD3 antibodies. The Examples demonstrate that mono-OKT3-Fab provides human CD3 copotentiation to enhance expansion of several classes of recall CD8 T cells with relevance to HCMV. First, mono-OKT3-Fab fulfilled the biochemical requirements to deliver copotentiation: binding to CD3 and inducing CD3Δc without initiating intrinsic signaling or interfering with TCR-antigen binding (FIGS. 1A-1F). Functional copotentiation was observed in recall assays where PBMCs from healthy blood donors were cultured with or without exogenous NLV and/or mono-OKT3-Fab. Enhanced expansion was observed both in A2/NLV-tetramer(+) and A2/NLV-tetramer(-) cells and in exog-NLV-bulk-nonresponsive donors (FIGS. 2A and 3A-3B). Expansion was impaired when using mono-UCHTI-Fab (FIGS. 3C-3D), which inhibits TCR-antigen binding and signaling (FIGS. 1A-1F), showing that Fab-CD3 was insufficient for copotentiation without TCR-antigen interaction. Mono-OKT3-Fab-mediated copotentiation was inhibited by anti-CD8 blocking antibody, showing that copotentiation depends on the tripartite CD8-TCR-HLA antigenic interaction (FIGS. 3E-3F). Strong NLV-reactive clones from A2/NLV-tetramer(+) donors (FIGS. 4A-4H and 5A-5C) and weak NLV-reactive clones from an HLA-A2+, A2/NLV-tetramer(-) donor were observed (74M, FIGS. 5A-5C). Together, these results support mono-OKT3-Fab copotentiation by enhancing HLA-dependent responses (FIG. 6). Copotentiation of peripheral blood CD8 T cells was also observed in classic recall assays with increased clonal expansion and effector function, showing that previously clonally expanded T cells are responsive to copotentiation (FIGS. 2A-2D and 3A-3F). Among them, public and private clones responding to immunodominant NLV:HLA-A2 antigen (FIGS. 4A-4H; Table 1) and clones for which NLV was a weak antigen (FIGS. 5A-5C) were observed.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A pharmaceutical composition comprising:
a monovalent anti-CD3 antibody, wherein the monovalent anti-CD3 antibody specifically binds to CD3, induces a conformational change in a CD3 complex (CD3Δc), does not initiate CD3 signaling, does not block interaction of a T cell receptor with a viral antigen, and does not block a T cell’s signaling response to the viral antigen; and
at least one of the viral antigen and a nucleic acid that encodes the viral antigen.

2. The pharmaceutical composition of claim 1, wherein the monovalent anti-CD3 antibody is selected from the group consisting of a Fab fragment of the anti-CD3 antibody, a Fab′ of the anti-CD3 antibody, a single chain Fv of the anti-CD3 antibody, a nanobody of the anti-CD3 antibody, and combinations thereof.

3. The pharmaceutical composition of claim 1, wherein the monovalent anti-CD3 antibody is a monovalent anti-human CD3 antibody.

4. The pharmaceutical composition of claim 1, wherein the monovalent anti-CD3 antibody is a humanized monovalent anti-CD3 antibody.

5. The pharmaceutical composition of claim 1, wherein the monovalent anti-CD3 antibody is selected from the group consisting of a monovalent OKT3 antibody, a monovalent UCHT1 antibody, a monovalent Hit3a antibody, a monovalent SP34-2 antibody, a monovalent SK7 antibody, a monovalent MEM-57 antibody, a monovalent Forlumab/28F11-AE/NI-0401 antibody, a monovalent Teplizumab/PRV-031/MGA031 antibody, a monovalent Visilizumab/HuM291 antibody, a monovalent Otelixizumab/ChAglyCD3/TRX4 antibody, and combinations thereof.

6. The pharmaceutical composition of claim 1, wherein the monovalent anti-CD3 antibody is a recombinant monovalent anti-CD3 antibody.

7. The pharmaceutical composition of claim 1, wherein the monovalent anti-CD3 antibody is a monovalent anti-CD3γε antibody.

8. The pharmaceutical composition of claim 7, wherein the monovalent anti-CD3γε antibody is a monovalent OKT3 antibody.

9. The pharmaceutical composition of claim 1, wherein the viral antigen is selected from the group consisting of a human cytomegalovirus (HCMV) antigen, an influenza antigen, a coronaviruse antigen, a rhinoviruse antigen, a human immunodeficiency virus (HIV) antigen, a hepatitis virus antigen, a polio virus antigen, a rabis virus antigen, a rubeola virus antigen, a variolla virus antigen, a mumps virus antigen, a papilloma virus antigen, a herpes zoster virus antigen, and combinations thereof.

10. The pharmaceutical composition of claim 1, wherein the pharmaceutical composition comprises the monovalent anti-CD3 antibody and the viral antigen.

11. The pharmaceutical composition of claim 1, wherein the pharmaceutical composition comprises the monovalent anti-CD3 antibody and the nucleic acid that encodes the viral antigen.

12. A method of treating a viral infection in a subject having or suspected of having the viral infection, the method comprising administering to the subject a pharmaceutical composition comprising a monovalent anti-CD3 antibody, wherein the monovalent anti-CD3 antibody specifically binds to CD3, induces a conformational change in a CD3 complex (CD3L1c), does not initiate CD3 signaling, does not block interaction of a T cell receptor with the viral antigen, and does not block a T cell’s signaling response to the viral antigen; and at least one of the viral antigen and a nucleic acid that encodes the viral antigen.

13. The method of claim 12, wherein the monovalent anti-CD3 antibody is selected from the group consisting of a monovalent OKT3 antibody, a monovalent UCHT1 antibody, a monovalent Hit3a antibody, a monovalent SP34-2 antibody, a monovalent SK7 antibody, a monovalent MEM-57 antibody, a monovalent Forlumab/28F11-AE/NI-0401 antibody, a monovalent Teplizumab/PRV-031/MGA031 antibody, a monovalent Visilizumab/HuM291 antibody, a monovalent Otelixizumab/ChAglyCD3/TRX4 antibody, and combinations thereof.

14. The method of claim 12, wherein the viral infection is a chronic viral infection.

15. The method of claim 14, wherein the chronic viral infection is selected from the group consisting of human cytomegalovirus infection, influenza infection, coronavirus infection, rhinoviruse infection, HIV infection, hepatitis virus infection, polio virus infection, rabis virus infection, rubeola virus infection, variolla virus infection, mumps virus infection, papilloma virus infection, and herpes zoster virus infection.

16. The method of claim 12, wherein the subject is a human.

17. A method for increasing an immune response against a viral antigen in a subject, the method comprising:
administering to the subject a pharmaceutical composition comprising a monovalent anti-CD3 antibody, wherein the monovalent anti-CD3 antibody specifically binds to CD3, induces a conformational change in a CD3 complex (CD3Δc), does not initiate CD3 signaling, does not block interaction of a T cell receptor with the viral antigen, and does not block a T cell’s signaling response to the viral antigen; and at least one of the viral antigen and a nucleic acid that encodes the viral antigen,
wherein the subject produces an immune response against the viral antigen.

18. The method of claim 17, wherein the subject is a human.

19. The method of claim 17, wherein the monovalent anti-CD3 antibody is selected from the group consisting of a monovalent OKT3 antibody, a monovalent UCHT1 antibody, a monovalent Hit3a antibody, a monovalent SP34-2 antibody, a monovalent SK7 antibody, a monovalent MEM-57 antibody, a monovalent Forlumab/28F11-AE/NI-0401 antibody, a monovalent Teplizumab/PRV-031/MGA031 antibody, a monovalent Visilizumab/HuM291 antibody, a monovalent Otelixizumab/ChAglyCD3/TRX4 antibody, and combinations thereof.

20. The method of claim 17, wherein the viral antigen is selected from the group consisting of a human cytomegalovirus (HCMV) antigen, an influenza antigen, a coronaviruse antigen, a rhinoviruse antigen, a human immunodeficiency virus (HIV) antigen, a hepatitis virus antigen, a polio virus antigen, a rabis virus antigen, a rubeola virus antigen, a variolla virus antigen, a mumps virus antigen, a papilloma virus antigen, a herpes zoster virus antigen, and combinations thereof.


SEQUENCE LISTING (53 sequences)

SEQ ID NO: 1 - protein, 9 aa, synthetic construct
NLVPMVATV

SEQ ID NO: 2 - protein, 16 aa, synthetic construct
CASSKVTGTGNYGYTF

SEQ ID NO: 3 - protein, 13 aa, synthetic construct
CASSLALNTEAFF

SEQ ID NO: 4 - protein, 15 aa, synthetic construct
CASSPSTGTIYGYTF

SEQ ID NO: 5 - protein, 16 aa, synthetic construct
CASSPITGQGAYGYTF

SEQ ID NO: 6 - protein, 13 aa, synthetic construct
CATFEEPNEKLFF

SEQ ID NO: 7 - protein, 15 aa, synthetic construct
CASSPWAYATDTQYF

SEQ ID NO: 8 - protein, 15 aa, synthetic construct
CASSYADRGAGELFF

SEQ ID NO: 9 - protein, 13 aa, synthetic construct
CASRDRENTEAFF

SEQ ID NO: 10 - protein, 14 aa, synthetic construct
CASSIDSPNTEAFF

SEQ ID NO: 11 - protein, 13 aa, synthetic construct
CARTGYEDTEAFF

SEQ ID NO: 12 - protein, 13 aa, synthetic construct
CASSRTSINEQFF

SEQ ID NO: 13 - protein, 13 aa, synthetic construct
CASSPEGGGGAFF

SEQ ID NO: 14 - protein, 16 aa, synthetic construct
CASSPTTGTGTYGYTF

SEQ ID NO: 15 - protein, 13 aa, synthetic construct
CASSLEGYTEAFF

SEQ ID NO: 16 - protein, 15 aa, synthetic construct
CASSPIAGYPHEQYF

SEQ ID NO: 17 - protein, 12 aa, synthetic construct
CASSPGTYGYTF

SEQ ID NO: 18 - protein, 11 aa, synthetic construct
CASSIMNEQFF

SEQ ID NO: 19 - protein, 13 aa, synthetic construct
CASSLAPPYEQYF

SEQ ID NO: 20 - protein, 15 aa, synthetic construct
CASSQGVGLGEKLFF

SEQ ID NO: 21 - protein, 15 aa, unidentified
CASSPRDNPNYGYTF

SEQ ID NO: 22 - protein, 11 aa, synthetic construct
CASSSVNEQFF

SEQ ID NO: 23 - protein, 15 aa, synthetic construct
CASSPKTGATYGYTF

SEQ ID NO: 24 - protein, 16 aa, synthetic construct
CASTPQTGTGYYGYTF

SEQ ID NO: 25 - protein, 13 aa, synthetic construct
CASGLGVNTEAFF

SEQ ID NO: 26 - protein, 12 aa, synthetic construct
CASRDGGYEQYF

SEQ ID NO: 27 - protein, 16 aa, unidentified
CASSSRTSGRFYEQYF

SEQ ID NO: 28 - protein, 17 aa, synthetic construct
CASSLDPSGRLGDEQYF

SEQ ID NO: 29 - protein, 14 aa, synthetic construct
CASSINYSNQPQHF

SEQ ID NO: 30 - protein, 16 aa, synthetic construct
CASSPKTGTGTYGYTF

SEQ ID NO: 31 - protein, 14 aa, synthetic construct
CSANQGGGNTEAFF

SEQ ID NO: 32 - protein, 16 aa, synthetic construct
CSVTLPQADGRYGYTF

SEQ ID NO: 33 - protein, 12 aa, synthetic construct
CASSSAYYGYTF

SEQ ID NO: 34 - protein, 16 aa, synthetic construct
CASSNPGGSSYYEQYF

SEQ ID NO: 35 - protein, 14 aa, synthetic construct
CASSQEPGNYGYTF

SEQ ID NO: 36 - protein, 15 aa, synthetic construct
CASSQTRGAGNTIYF

SEQ ID NO: 37 - protein, 13 aa, synthetic construct
CASSLGQAYEQYF

SEQ ID NO: 38 - protein, 14 aa, synthetic construct
CASRQGFPGNEQFF

SEQ ID NO: 39 - protein, 15 aa, synthetic construct
CASRSLRDLNTEAFF

SEQ ID NO: 40 - protein, 17 aa, synthetic construct
CASSQVPDSDCNQPQHF

SEQ ID NO: 41 - protein, 18 aa, synthetic construct
CASSEEWGTSGGANEQFF

SEQ ID NO: 42 - protein, 14 aa, synthetic construct
CASCSTTGYETQYF

SEQ ID NO: 43 - protein, 15 aa, unidentified
CASSLAETENTEAFF

SEQ ID NO: 44 - protein, 16 aa, synthetic construct
CASSSRFGTGTHEQYF

SEQ ID NO: 45 - protein, 19 aa, synthetic construct
CASSQDYPPAGGTNNEQFF

SEQ ID NO: 46 - protein, 15 aa, synthetic construct
CSVEDEDSRTDTQYF

SEQ ID NO: 47 - protein, 17 aa, synthetic construct
CSAGRGIKTGRSETQYF

SEQ ID NO: 48 - protein, 15 aa, synthetic construct
CASSRQRTYTGELFF

SEQ ID NO: 49 - protein, 15 aa, synthetic construct
CASSVAGGLQETQYF

SEQ ID NO: 50 - protein, 12 aa, synthetic construct
CASSLVGVEAFF

SEQ ID NO: 51 - protein, 12 aa, synthetic construct
CASSLQTGVAFF

SEQ ID NO: 52 - protein, 8 aa, synthetic construct
SIINFEKL

SEQ ID NO: 53 - protein, 8 aa, synthetic construct
SSIEFARL